(19) United States
(12) Patent Application Publication
(10) Pub. No.: US 2023/0277568 A1
(43) Pub. Date: Sep. 7, 2023

(54) METHOD OF TREATMENT FOR PSILOCYBIN OR PSILOCIN INFUSION

(71) Applicants: Eleusis Therapeutics US, Inc., New York, NY (US); BOARD OF SUPERVISORS OF LOUISIANA STATE UNIVERSITY AND AGRICULTURAL AND MECHANICAL COLLEGE, Baton Rouge, LA (US)

(72) Inventors: Ryan Steven BRIDGES, Orlando, FL (US); J. Cannon CLIFTON, Helotes, TX (US); John Bryan CLIFTON, Helotes, TX (US); Neiloufar FAMILY, Brooklyn, NY (US); Emeline L. MAILLET, New Orleans, LA (US); Shlomi RAZ, Brooklyn, NY (US); David E. NICHOLS, Chapel Hill, NC (US); Charles D. NICHOLS, New Orleans, LA (US)

(21) Appl. No.: 18/015,277

(22) PCT Filed: Jul. 12, 2021
(86) PCT No.: PCT/US2021/041321

§ 371 Date: Jan. 9, 2023

Related U.S. Application Data
(60) Provisional application No. 63/091,068, filed on Oct. 13, 2020.
(60) Provisional application No. 63/050,428, filed on Jul. 10, 2020.

(51) Int. Cl. A61K 31/675; A61P 25/24; A61P 25/22; A61P 25/04; A61K 9/00; A61K 31/4045; A61K 31/5513; A61K 45/06; A61P 25/00

(52) U.S. Cl. CPC A61K 31/675; A61P 25/24; A61P 25/22; A61P 25/04; A61K 9/0019; A61K 31/4045; A61K 31/5513; A61K 45/06; A61P 25/00

(57) ABSTRACT

Methods for treatment for patients suffering from disease or condition are contemplated as including an administration of an intravenous infusion of a pharmaceutically effective amount of psilocybin or psilocin. The intravenous infusion of psilocybin or psilocin may include an additional compound such as a benzodiazepine, preferably lorazepam, administered via a continuous intra-venous infusion. Such methods may be seen to better alleviate the symptoms of psychological conditions, neurological injuries, pain, or inflammatory condition, and may result in reduced need for other medications.

### BACKGROUND OF THE INVENTION

Significant interest in the therapeutic application of psilocybin has developed, based upon evidence of possible therapeutic effects in a wide array of clinical applications, including psychiatric conditions, pain disorders, and neurological conditions. However, harnessing the full therapeutic utility of psilocybin requires new methods to mitigate side effects, enhance safety and efficacy, reduce or prolong treatment time required for therapeutic effect, and reduce interpatient variability in treatment response associated with psilocybin administration.

### SUMMARY OF THE INVENTION

In a first aspect the disclosure provides a method of treating a disease or condition in a subject in need thereof, the method including intravenously administering to the subject a free base equivalent of from 1 mg to 15 mg (e.g., 2±1 mg, 3±1 mg, 4±1 mg, 5±1 mg, 6±1 mg, 7±1 mg, 8±1, 9±1 mg, 10±1 mg, 11±1 mg, 12±1 mg, 13±1 mg, or 14±1 mg) of psilocin, or a pharmaceutically acceptable salt thereof, over a period of between 1 minute and 60 minutes (e.g., over a period of about 1 to 2 minutes, 2 to 5 minutes, 3 to 7 minutes, 5 to 10 minutes, 10 to 15 minutes, 15 to 20 minutes, 20 to 30 minutes, or 30 minutes to 60 minutes). In particular embodiments, a free base equivalent of from 4 mg to 15 mg (e.g., 6±2 mg, 8±2 mg, 10±2 mg, or 13±2 mg) of psilocin, or a pharmaceutically acceptable salt thereof, is administered to the subject over a period of 20 to 60 minutes (e.g., over a period of about 25±5 minutes, 30±5 minutes, 35±5 minutes, 40±5 minutes, 45±5 minutes, 50±5 minutes, or 55±5 minutes). In some embodiments, a free base equivalent of 5.0±1.0 mg of psilocin, or a pharmaceutically acceptable salt thereof, is administered over a period of 20 to 30 minutes. In certain embodiments, a free base equivalent of 7.5±1.0 mg of psilocin, or a pharmaceutically acceptable salt thereof, is administered over a period of 20 to 45 minutes (e.g., over a period of about 25±5 minutes, 30±5 minutes, 35±5 minutes, or 40±5 minutes). In some embodiments, a free base equivalent of 10.0±2.0 mg of psilocin, or a pharmaceutically acceptable salt thereof, is administered over a period of 30 to 60 minutes (e.g., over a period of about 35±5 minutes, 40±5 minutes, 45±5 minutes, 50±5 minutes, or 55±5 minutes). In particular embodiments, a free base equivalent of from 1 mg to 5 mg (e.g., 2±1 mg, 3±1 mg, or 4±1 mg) of psilocin, or a pharmaceutically acceptable salt thereof, is administered to the subject over a period of 2 to 20 minutes (e.g., over a period of about 3±1 minutes, 4±1 minutes, 5±1 minutes, 7±2 minutes, 10±2 minutes, 12±2 minutes, or 15±5 minutes). In some embodiments, a free base equivalent of 4.0±1.0 mg of psilocin, or a pharmaceutically acceptable salt thereof, is administered over a period of 5 to 20 minutes (e.g., over a period of about 7±2 minutes, 10±2 minutes, 12±2 minutes, or 15±5 minutes). In certain embodiments, a free base equivalent of 3.0±1.0 mg of psilocin, or a pharmaceutically acceptable salt thereof, is administered over a period of 5 to 15 minutes (e.g., over a period of about 7±2 minutes, 10±2 minutes, 12±2 minutes, or 13±2 minutes). In some embodiments a free base equivalent of 2.0±1.0 mg of psilocin, or a pharmaceutically acceptable salt thereof, is administered over a period of 2 to 10 minutes (e.g., over a period of about 3±1 minutes, 4±1 minutes, 5±1 minutes, 6±2 minutes, or 8±2 minutes). In any of the above methods, the intravenously administering can be a continuous infusion over the period.

In another aspect the invention features a method of treating a disease or condition in a subject in need thereof, the method including intravenously administering to the subject a pharmacologically effective amount of psilocybin or psilocin, or a pharmaceutically acceptable salt thereof, and a benzodiazepine, each in an amount that together is effective for the treatment of the disease or condition. In particular embodiments, the benzodiazepine is lorazepam or diazepam.

In a related aspect the disclosure provides a method of treating a disease or condition in a subject in need thereof, the method including administering to the subject a timed intravenous infusion of psilocybin or psilocin, or a pharmaceutically acceptable salt thereof, wherein (i) the timed intravenous infusion is administered at a free base equivalent rate of between 1 mg/hr and 15 mg/hr (e.g., 2±1 mg/hr, 3±1 mg/hr, 4±1 mg/hr, 5±1 mg/hr, 6±1 mg/hr, 7±1 mg/hr, 8±1 mg/hr, 9±1 mg/hr, 10±1 mg/hr, 11±1 mg/hr, 12±1 mg/hr, 13±1 mg/hr, and 14±1 mg/hr) of psilocin or an equimolar equivalent of psilocybin for a period of time between 10 minutes and 60 minutes (e.g., over a period of about 15±5 minutes, 20±5 minutes, 25±5 minutes, 30±5 minutes, 35±5 minutes, 40±5 minutes, 45±5 minutes, 50±5 minutes, or 55±5 minutes); or (ii) the timed intravenous infusion is administered at a free base equivalent rate of between 15 mg/hr and 60 mg/hr (e.g., 17±2 mg/hr, 15±3 mg/hr, 20±5 mg/hr, 25±5 mg/hr, 30±5 mg/hr, 35±5 mg/hr, 40±5 mg/hr, 45±5 mg/hr, and 50±10 mg/hr) of psilocin or an equimolar equivalent of psilocybin for a period of time between 2 minutes and 10 minutes (e.g., over a period of about 3±1 minutes, 4±2 minutes, 5±2 minutes, 7±2 minutes, or 8±2 minutes). In some embodiments, the pharmacologically effective amount of psilocybin or psilocin is administered as a saline solution. In one embodiment, the method includes intravenously administering to the subject a continuous infusion of psilocybin, or a pharmaceutically acceptable salt thereof. In another embodiment, the method includes intravenously administering to the subject a continuous infusion of psilocin, or a pharmaceutically acceptable salt thereof.

In some embodiments, the method includes further administering to the patient a pharmacologically effective amount of an antiemetic agent. In certain embodiments, the antiemetic agent includes a non-selective 5-HT antagonist, 5-HT3 receptor antagonist, 5-HT4 receptor agonist, CB1 agonist, D2 receptor antagonist, D3 receptor antagonist, GABA receptor agonist, H1 receptor antagonist, muscarinic acetylcholine receptor antagonist, NK1 receptor antagonist, or a combination thereof. In particular embodiments, the anti-emetic ondansetron is intravenously infused.

In some embodiments, the intravenous infusion includes a pharmacologically effective amount of a benzodiazepine. In certain embodiments, the benzodiazepine is a 1,4-benzodiazepine, 1,5-benzodiazepine, 2,3-benzodiazepine, triazolobenzodiazepine, imidazobenzodiazepine, oxazolobenzodiazepine, thienodiazepine, thienotriazolodiazepine, thienobenzodiazepine, pyridodiazepine, pyridotriazolodiazepine, pyrralodiazepine, tetrahydroisoquinobenzodiazepine, a benzodiazepine prodrug, or a combination thereof. In particular embodiments, the benzodiazepine is lorazepam. In some embodiments, the benzodiazepine is administered in a dosage of between 2 mg and 10 mg (e.g., 2±1 mg, 3±1 mg, 4±1 mg, 5±1 mg, 6±1 mg, 7±1 mg, 8±1, 9±1 mg, and 10±1 mg). In particular embodiments, the lorazepam is administered in a dosage between 2 mg and 4 mg (e.g., 2±1 mg, 3±1 mg, and 4±1 mg). In certain embodiments, the benzodiazepine is diazepam. In particular embodiments, the diazepam is administered in a dosage between 5 mg and 10 mg (e.g., 5±1 mg, 6±1 mg, 7±1 mg, 8±1, 9±1 mg, and 10±1 mg). In certain embodiments, the ratio of psilocybin or psilocin to benzodiazepine is between 100:1 and 10:1 (e.g., 90:1, 80:1, 0:1, 60:1 50:1, 40:1, 30:1, 20:1, and 10:1) by weight.

In some embodiments, the intravenous infusion includes a pharmacologically effective amount of an anesthetic, a sedative, an antiemetic, an anticonvulsant, an antidepressant, an antimigraine, an antipsychotic, an anxiolytic, and/or an antiparkinson agent. In particular embodiments, intravenous infusion includes ondansetron or a pharmaceutically acceptable salt thereof.

In some embodiments, the method further includes administering to the patient a preparation including a pharmacologically effective amount of an anti-inflammatory agent. In particular embodiments, the administration of the preparation is an intravenous infusion of ketorolac or pharmaceutically acceptable salt thereof. In other embodiments, the administration of the preparation is an intramuscular infusion of a pharmacologically effective amount of a triptan or a pharmaceutically acceptable salt thereof. In particular embodiments, the preparation includes sumatriptan or a pharmaceutically acceptable salt thereof.

In certain embodiments, the rate of administration of the intravenous infusion including a pharmacologically effective amount of psilocybin or psilocin is configured to vary during the period of administration. In some embodiments, the subject's intensity of experience is monitored (e.g., by a nurse, physician, or monitoring measures of stress, such as blood pressure or hear rate). In particular embodiments, the rate of administration of the intravenous infusion including a pharmacologically effective amount of psilocybin or psilocin is adjusted in response to the subject's intensity of experience.

In certain embodiments, the patient's plasma concentration of psilocin is monitored.

In some embodiments, the intravenous infusion including a pharmacologically effective amount of psilocybin or psilocin is administered at least twice over the course of a month. In some embodiments, the intravenous infusion including a pharmacologically effective amount of psilocybin or psilocin is administered between 2 and 10 times (e.g., 3, 4, 5, 6, 7, 8, and 9 times) over the course of a year.

In particular embodiments, the timed intravenous infusion of the pharmacologically effective amount of psilocybin or psilocin is administered at a free base equivalent rate of between 4 mg/hr and 15 mg/hr (e.g., 5±1 mg/hr, 6±1 mg/hr, 7±1 mg/hr, 8±1 mg/hr, 9±1 mg/hr, 10±1 mg/hr, 11±1 mg/hr, 12±1 mg/hr, 13±1 mg/hr, or 14±1 mg/hr) of psilocin or an equimolar equivalent of psilocybin over a period of time of between 10 minutes and 60 minutes (e.g., over a period of about 15±5 minutes, 20±5 minutes, 25±5 minutes, 30±5 minutes, 35±5 minutes, 40±5 minutes, 45±5 minutes, 50±5 minutes, or 55±5 minutes). In some embodiments, a free base equivalent of 4.0±0.5 mg or 5.0±0.5 mg of psilocin or an equimolar equivalent of psilocybin is administered over a period of 20 to 60 minutes (e.g., over a period of about 25±5 minutes, 30±5 minutes, 35±5 minutes, 40±5 minutes, 45±5 minutes, 50±5 minutes, or 55±5 minutes). In certain embodiments, a free base equivalent of 10.0±1.0 mg of psilocybin or psilocin is administered over a period of 45 to 60 minutes (e.g., over a period of about 50±5 minutes, or 55±5 minutes).

In particular embodiments, the timed intravenous infusion of the pharmacologically effective amount of psilocybin or psilocin is administered at a free base equivalent rate of between 15 mg/hr and 30 mg/hr (e.g., 17±2 mg/hr, 15±3 mg/hr, 20±5 mg/hr, or 25±5 mg/hr) of psilocin or an equimolar equivalent of psilocybin over a period of time of between 2 minutes and 10 minutes (e.g., over a period of about 3±1 minutes, 4±2 minutes, 5±2 minutes, 7±2 minutes, or 8±2 minutes). In some embodiments, a free base equivalent of 1.0±0.5 mg or 2.0±0.5 mg of psilocin or an equimolar equivalent of psilocybin is administered over a period of 2 to 10 minutes (e.g., over a period of about 3±1 minutes, 4±2 minutes, 5±2 minutes, 7±2 minutes, or 8±2 minutes). In certain embodiments, a free base equivalent of 4.0±0.5 mg of psilocin or an equimolar equivalent of psilocybin is administered over a period of 5 to 10 minutes (e.g., over a period of about 6±1 minutes, 7±1 minutes, 8±1 minutes, or 9±1 minutes). In some embodiments, a free base equivalent of 5.0±0.5 mg of psilocin or an equimolar equivalent of psilocybin is administered over a period of 10 to 15 minutes (e.g., over a period of about 11±1 minutes, 12±1 minutes, 13±1 minutes, or 14±1 minutes).

In some embodiments, the disease or condition being treated is a psychological condition. In certain embodiments, the psychological condition is evaluated 1-8 weeks (e.g., 2, 3, 4, 5, 6, and 7 weeks) after treatment. In some embodiments, the psychological condition is evaluated 1 week after treatment. In some embodiments, the psychological condition is evaluated 4 weeks after treatment. In certain embodiments, the psychological condition is depression, anxiety, addiction, post-traumatic stress disorder, an eating disorder, or compulsive behavior. In particular embodiments, the psychological condition is depression. In some embodiments, the depression is evaluated using the Hamilton Depression Rating Scale (HAM-D). In certain embodiments, the HAM-D score decreases compared to the score before treatment. In particular embodiments, the HAM-D score decreases by 50% compared to the score before treatment. In some embodiments, the depression is evaluated using the Beck Depression Inventory Scale (BDI). In certain embodiments, the BDI score decreases compared to the score before treatment. In particular embodiments, the BDI score decreases by 50% compared to the score before treatment. In some embodiments, the depression is evaluated using the Quick Inventory of Depressive Symptomatology (QIDS) score. In certain embodiments, the AIDS score decreases compared to the score before treatment. In some embodiments, the AIDS score decreases by 50% compared to the score before treatment. In certain embodiments, the depression is evaluated using a Montgomery-Asberg Depression Rating Scale. In some embodiments, the Montgomery-Asberg Depression Rating Scale score decreases compared to the score before treatment. In certain embodiments, the Montgomery-Asberg Depression Rating Scale score decreases by 50% compared to the score before treatment. In particular embodiments, the Montgomery-Asberg Depression Rating Scale score is less than 10 after treatment. In some embodiments, the psychological condition is anxiety. In certain embodiments, the anxiety is end of life anxiety, or anxiety of a subject receiving palliative care.

In some embodiments, the disease or condition is a neurological injury, an inflammatory condition, or pain, (e.g., chronic pain). In particular embodiments, the disease or condition is an inflammatory condition. In certain embodiments, the inflammatory condition is lung inflammation, neuroinflammation, rheumatoid arthritis, atherosclerosis, psoriasis, type II diabetes, inflammatory bowel disease, Crohn's disease, multiple sclerosis, and/or septicemia. In certain embodiments, the inflammatory condition is chronic obstructive pulmonary disease (COPD)), or Alzheimer's disease. In some embodiments, the disease or condition is a neurological injury. In particular embodiments, the neurological injury is a stroke, a traumatic brain injury, or a spinal cord injury. In some embodiments, the disease or condition is pain, including acute pain and a chronic pain condition. In particular embodiments, the chronic pain condition results from post-operative pain, tension headaches, chronic lower back pain, fibromyalgia, nephropathy, multiple sclerosis, shingles, complex regional pain syndrome, cephalic pain, or sciatica. In some embodiments, the chronic pain condition results from trigeminal autonomic cephalalgia. In certain embodiments, trigeminal autonomic cephalalgia is selected from the group consisting of episodic and chronic cluster headache (CH), episodic and chronic paroxysmal hemicrania (PH), and short-lasting unilateral neuralgiform headache attacks with conjunctival injection and tearing (SUNCT). In particular embodiments, the trigeminal autonomic cephalalgia is episodic or chronic CH. In certain embodiments, the method further includes administering to the patient one or more medications for pain relief, including morphine, hydromorphone, hydrocodone, meperidine, and fentanyl. In particular embodiments, the method of treating pain, neurological injury, or an inflammatory condition can include a continuous intravenous infusion of the pharmacologically effective amount of psilocybin or psilocin that is administered at a free base equivalent rate of between 1 mg/hr and 15 mg/hr (e.g., 2±1 mg/hr, 3±1 mg/hr, 4±1 mg/hr, 5±1 mg/hr, 6±1 mg/hr, 7±1 mg/hr, 8±1 mg/hr, 9±1 mg/hr, 10±1 mg/hr, 11±1 mg/hr, 12±1 mg/hr, 13±1 mg/hr, or 14±1 mg/hr) of psilocin or an equimolar equivalent of psilocybin over a period of time of between 10 minutes and 60 minutes (e.g., over a period of about 15±5 minutes, 20±5 minutes, 25±5 minutes, 30±5 minutes, 35±5 minutes, 40±5 minutes, 45±5 minutes, 50±5 minutes, or 55±5 minutes).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A is a graph showing a simulation for pharmacokinetic data measured as the concentration of psilocin in the plasma in ng/mL for a subject dosed with 0.5 mg, 1 mg, 2 mg, 6 mg, or 8 mg of psilocybin over a 30 second infusion.

FIG. 1B is a graph showing a simulation for pharmacodynamic data measured in terms of intensity of acute subjective effects for a subject dosed with 0.5 mg, 1 mg, 2 mg, 6 mg, or 8 mg of psilocybin over a 30 second infusion.

FIG. 2A is a graph showing a simulation for pharmacokinetic data measured as the concentration of psilocin in the plasma in ng/mL for a subject dosed with 0.5 mg, 1 mg, 2 mg, 6 mg, or 8 mg of psilocybin over a 30 minute infusion.

FIG. 2B is a graph showing a simulation for pharmacodynamic data measured in terms of intensity of acute subjective effects for a subject dosed with 0.5 mg, 1 mg, 2 mg, 6 mg, or 8 mg of psilocybin over a 30 minute infusion.

FIG. 3A is a graph showing a simulation for pharmacokinetic data measured as the concentration of psilocin in the plasma in ng/mL for a subject dosed with 0.5 mg, 1 mg, 2 mg, 6 mg, or 8 mg of psilocybin over a 1 hour infusion.

FIG. 3B is a graph showing a simulation for pharmacodynamic data measured in terms of intensity of acute subjective effects for a subject dosed with 0.5 mg, 1 mg, 2 mg, 6 mg, or 8 mg of psilocybin over a 1 hour infusion.

FIG. 4 is a graph showing a simulation for pharmacokinetic data measured as the psilocin concentration in the plasma in ng/mL for a subject dosed with 1 mg, 2 mg, 4 mg, 5 mg, and 10 mg of psilocin over a 2 minute infusion. Overlaid as horizontal solid lines are 10 and 20 ng/mL concentrations that represent an exemplary target therapeutic level. Further overlaid in a dashed line are the simulated pharmacokinetics following 25 mg of psilocybin orally administered.

FIG. 5 is a graph showing a simulation for pharmacokinetic data measured as the psilocin concentration in the plasma in ng/mL for a subject dosed with 1 mg, 2 mg, 4 mg, 5 mg, and 10 mg of psilocin over a 10 minute infusion. Overlaid as horizontal solid lines are 10 and 20 ng/mL concentrations that represent an exemplary target therapeutic level. Further overlaid in a dashed line are the simulated pharmacokinetics following 25 mg of psilocybin orally administered.

FIG. 6 is a graph showing a simulation for pharmacokinetic data measured as the psilocin concentration in the plasma in ng/mL for a subject dosed with 1 mg, 2 mg, 4 mg, 5 mg, and 10 mg of psilocin over a 20 minute infusion. Overlaid as horizontal solid lines are 10 and 20 ng/mL concentrations that represent an exemplary target therapeutic level. Further overlaid in a dashed line are the simulated pharmacokinetics following 25 mg of psilocybin orally administered.

FIG. 7 is a graph showing a simulation for pharmacokinetic data measured as the psilocin concentration in the plasma in ng/mL for a subject dosed with 1 mg, 2 mg, 4 mg, 5 mg, and 10 mg of psilocin over a 30 minute infusion. Overlaid as horizontal solid lines are 10 and 20 ng/mL concentrations that represent an exemplary target therapeutic level. Further overlaid in a dashed line are the simulated pharmacokinetics following 25 mg of psilocybin orally administered.

FIG. 8 is a graph showing a simulation for pharmacokinetic data measured as the psilocin concentration in the plasma in ng/mL for a subject dosed with 1 mg, 2 mg, 4 mg, 5 mg, and 10 mg of psilocin over a 45 minute infusion. Overlaid as horizontal solid lines are 10 and 20 ng/mL concentrations that represent an exemplary target therapeutic level. Further overlaid in a dashed line are the simulated pharmacokinetics following 25 mg of psilocybin orally administered.

FIG. 9 is a graph showing a simulation for pharmacokinetic data measured as the psilocin concentration in the plasma in ng/mL for a subject dosed with 1 mg, 2 mg, 4 mg, 5 mg, and 10 mg of psilocin over a 60 minute infusion. Overlaid as horizontal solid lines are 10 and 20 ng/mL concentrations that represent an exemplary target therapeutic level. Further overlaid in a dashed line are the simulated pharmacokinetics following 25 mg of psilocybin orally administered.

FIGS. 10A-10C is a series of bar graphs depicting the effect of psilocin and lorazepam in a rat model of depression as described in Example 16: SAL: Saline injected WKY rats; PSI: 1 mg/kg of psilocin was injected as a bolus dose into the tail vein of WKY rats; and P+L: 1.8 mg/kg of lorazepam was injected 30 i.p. minutes prior to 1 mg/kg tail vein injection of psilocin. Conclusions: (i) A single bolus of i.v. administered psilocin is as efficacious in producing long-lasting antidepressant-like behaviors in our model as a single i.p. administration of psilocybin of the same dose; (ii) lorazepam only had a slight, but significant effect, on one outcome measure to reduce efficacy (Immobility); and (iii) The P+L combination produces long-lasting antidepressant-like effects in our model.

### DEFINITIONS

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

As used herein, the term “about” refers to a value that is within 10% above or below the value being described.

As used herein, the terms “acute stress disorder” and “ASD” refer to a condition that arises as a response to a stressful event or situation of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Like PTSD, acute stress disorder is an anxiety disorder that involves a very specific reaction following exposure to a traumatic event or stressor. However, the duration of acute stress disorder is shorter than that for PTSD, such that the symptoms are present for at least one, two, or three days, but no more than four, five, or six weeks. For individuals exhibiting symptoms persisting for a longer period of time, a diagnosis of PTSD may be warranted.

The term “administration” or “administering” refers to a method of giving a dosage of a compound or pharmaceutical composition to a subject.

As used herein, the term “continuous infusion” refers to an infusion of a drug (e.g., psilocybin, or psilocin, or pharmaceutically acceptable salts thereof) such that the plasma concentration of the drug and/or metabolite (e.g., psilocin) does not vary by more than ±10% for at least 15 minutes, unless the rate of infusion is altered in response to the subject's intensity rating.

As used herein, the terms “dosage” and “unit dose” when used in reference to a therapeutic composition refer to physically discrete units suitable as unitary dosage for the subject, each unit containing a predetermined quantity of active material calculated to produce the desired therapeutic effect in association with the required diluent, i.e., carrier, or vehicle.

By “dysthymia” or “dysthymic disorder” is meant a chronically depressed mood that occurs for most of the day, more days than not, for at least two years. In children and adolescents, the mood may be irritable rather than depressed, and the required minimum duration is one year. During the two year period (one year for children or adolescents), any symptom-free intervals last no longer than 2 months. During periods of depressed mood, at least two of the following additional symptoms are present: poor appetite or overeating, insomnia or hypersomnia, low energy or fatigue, low self-esteem, poor concentration, or difficulty making decisions, and feelings of hopelessness. The symptoms cause clinically significant distress or impairment in social, occupational (or academic), or other important areas of functioning. The diagnosis of dysthymia is not made if: the individual has ever had a manic episode, a mixed episode, a hypomanic episode; has ever met the criteria for a cyclothymic disorder; the depressive symptoms occur exclusively during the course of a chronic psychotic disorder (e.g., schizophrenia); or if the disturbance is due to the direct physiological effects of a substance or a general medical condition. After the initial two-years of dysthymic disorder, major depressive episodes may be superimposed on the dysthymic disorder (“double depression”). Diagnostic and Statistical Manual of Mental Disorders (OSM IV), American Psychiatric Press, 4th Edition, 1994. Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, Va.; American Psychiatric Association, 2013).

By “free base equivalent” is meant an amount corresponding to a free base equivalent in a mass of a psilocin salt form. For example, a free base equivalent of 1 mg of psilocin is equal to 1 mg of psilocin in its free base form and equal to 1.17 mg of psilocin in its hydrochloride salt form (e.g., 1.0×(240.728/204.27) to account for the mass contribution of the hydrochloride).

As used herein, the term “generalized anxiety disorder” refers to a condition characterized by excessive anxiety and worry (i.e., apprehensive expectation). Typically, the excessive anxiety and worry occur on more days than not for a period of time (e.g., one, two, three, or four months or more). The anxiety and worry can be associated with (i) restlessness, feeling keyed up, or on edge; and/or (ii) muscle tension. The anxiety and worry can be associated with (a) a marked avoidance of situations in which a negative outcome could occur; (b) a marked time and effort preparing for situations in which a negative outcome could occur; (c) a marked procrastination in behavior or decision-making due to worries; and (d) repeatedly seeking reassurance due to worries. The anxiety, worry, or physical symptoms can cause clinically significant distress or impairment in social, occupational, or other important areas of functioning in many, but not necessarily all individuals with GAD.

As used herein, the term “intensity rating,” “intensity of experience,” and “intensity of acute subjective effects” refer to the intensity of an experience a subject has after being administered a particular drug measured on a scale from 1 to 10 by subjects. An intensity rating of less than 2 may indicate that it is safe for a patient to leave the clinic. The intensity rating described by the subject is used to determine whether the infusion rate of a drug should be increased, decreased, or remain the same.

As used herein, the terms “obsessive compulsive disorder,” “OCD,” and “anxiety and obsessive-compulsive spectrum disorders” refer to a condition characterized by obsessions and/or compulsions. Obsessions are recurrent and persistent thoughts, urges, or images that are experienced, at some time during the disturbance, as intrusive and unwanted and that usually cause marked anxiety or distress in which the obsessed individual attempts to ignore or suppress such thoughts, urges, or images, or to neutralize them with some other thought or action (i.e., by performing a compulsion). Compulsions are repetitive behaviors (e.g., hand washing, ordering, checking) or mental acts (e.g., praying, counting, repeating words silently) that the person feels driven to perform in response to an obsession, or according to rules that must be applied rigidly. The behaviors or mental acts are aimed at preventing or reducing anxiety or distress, or preventing some dreaded event or situation; however, these behaviors or mental acts either are not connected in a realistic way with what they are designed to neutralize or prevent, or are clearly excessive. Typically the obsessions or compulsions are time consuming (for example, take more than 1 hour a day), or cause clinically significant distress or impairment in social, occupational, or other important areas of functioning.

As used herein, the term “pharmaceutically acceptable salt” refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic or inorganic acid.

As used herein, the term “panic disorder” refers to a condition characterized by recurrent and unexpected panic attacks. Panic disorder includes both panic disorder with agoraphobia and panic disorder without agoraphobia. Subjects with this condition can exhibit one or both of the following: (i) a persistent concern or worry about additional panic attacks or their consequences (e.g., losing control, having a heart attack, going crazy); and/or (ii) significant maladaptive change in behavior related to the attacks (e.g., behaviors designed to avoid having panic attacks), which may include agoraphobic avoidance.

As used herein, the terms “pharmacologically effective amount,” “therapeutically effective amount,” and the like, when used in reference to a therapeutic composition, refer to a quantity sufficient to, when administered to the subject, including a mammal, for example a human, effect beneficial or desired results, such as clinical results. For example, in the context of treating depression, described herein, these terms refer to an amount of the composition sufficient to achieve a treatment response as compared to the response obtained without administration of the composition. The quantity of a given composition described herein that will correspond to such an amount may vary depending upon various factors, such as the given agent, the pharmaceutical formulation, the route of administration, the type of disease or disorder, the identity of the subject (e.g., age, sex, weight) or host being treated, and the like. An “effective amount,” “pharmacologically effective amount,” or the like, of a composition of the present disclosure, also include an amount that results in a beneficial or desired result in a subject as compared to a control (e.g., a decrease in the score on the Montgomery-Asberg Depression Rating Scale).

As used herein, the terms “post traumatic stress disorder” and “PTSD” refer to a condition that arises as a delayed and/or protracted response to a stressful event or situation (either short- or long-lasting) of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Predisposing factors such as personality traits (e.g., compulsive, asthenic) or previous history of neurotic illness may lower the threshold for the development of the condition or aggravate its course, but they are neither necessary nor sufficient to explain its occurrence. PTSD is a less frequent and more enduring consequence of psychological trauma than the more frequently seen acute stress response. PTSD has been recognized in the past as railway spine, stress syndrome, shell shock, battle fatigue, traumatic war neurosis, and post-traumatic stress syndrome. Diagnostic symptoms include re-experiencing original trauma(s), by means of flashbacks or nightmares; avoidance of stimuli associated with the trauma; and increased arousal, such as difficulty falling or staying asleep, anger, and hypervigilance. Formal diagnostic criteria (DSM-V, DSM-IV, and/or ICD-9) require that the symptoms last more than one month and cause significant impairment in social, occupational, or other important areas of functioning (e.g., problems with work and/or relationships). Formal diagnostic criteria can include: (i) intrusion symptoms that are associated with the traumatic event (e.g., (a) spontaneous or cued recurrent, involuntary, and intrusive distressing memories of the traumatic event; (b) recurrent distressing dreams in which the content and/or affect of the dream is related to the event; (c) dissociative reactions (e.g., flashbacks) in which the individual feels or acts as if the traumatic event were recurring (such reactions may occur on a continuum, with the most extreme expression being a complete loss of awareness of present surroundings; (d) intense or prolonged psychological distress at exposure to internal or external cues that symbolize or resemble an aspect of the traumatic event; and/or (e) marked physiological reactions to reminders of the traumatic event); (ii) persistent avoidance of stimuli associated with the traumatic event (e.g., (a) thoughts, feelings, or physical sensations that arouse recollections of the traumatic event; (b) activities, places, physical reminders, or times (e.g., anniversary reactions) that arouse recollections of the traumatic event; and/or (c) people, conversations, or interpersonal situations that arouse recollections of the traumatic event); (iii) negative alterations in cognitions and mood that are associated with the traumatic event (e.g., (a) inability to remember an important aspect of the traumatic event (typically dissociative amnesia); (b) persistent and exaggerated negative expectations about one's self, others, or the world; (c) persistent distorted blame of self or others about the cause or consequences of the traumatic event; (d) pervasive negative emotional state (e.g., fear, horror, anger, guilt, or shame); (e) markedly diminished interest or participation in significant activities; (f) feeling of detachment or estrangement from others; and/or (g) persistent inability to experience positive emotions (e.g., unable to have loving feelings, psychic numbing); and (iv) alterations in arousal (i.e., hyperarousal) and reactivity that are associated with the traumatic event (e.g., (a) irritable, angry, or aggressive behavior; (b) reckless or self-destructive behavior; (c) hypervigilance; (d) exaggerated startle response; (e) problems with concentration; and/or (f) sleep disturbance (e.g., difficulty falling or staying asleep, or restless sleep)). Formal diagnostic criteria can further include that the duration of disturbance is more than a certain period of time (e.g., one month, three months, or six months) and that the disturbance causes clinically significant distress or impairment in social, occupational, or other important areas of functioning. In a small proportion of patients the condition may show a chronic course over many years and a transition to an enduring personality change. The three main symptoms associated with PTSD are (1) “reliving” the traumatic event, such as flashbacks, nightmares, intrusive thoughts and recollections, (2) avoidance behaviors and emotional numbing, and (3) hypersensitivity such as an inability to sleep, anxious feelings, overactive startle response, hyperarousal, hypervigilance, irritability, and outbursts of anger.

As used herein, the terms “psychological disorder” and “psychological condition” refer to a condition characterized by a disturbance in one's emotional or behavioral regulation that reflects a dysfunction in the psychological, biological, or developmental processes underlying mental function. Psychological disorders include, but are not limited to depressive disorders (major depression, treatment resistant depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcoholism, tobacco abuse, or drug abuse)), eating disorders (e.g., anorexia nervosa, bulimia nervosa, and binge eating disorder) and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms (e.g., back pain, fibromyalgia, migraines, and chronic fatigue syndrome). Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

As used herein, the terms “social phobia” and “social anxiety disorder” refer to a condition characterized by fear or anxiety associated with one or more social situations. Subjects with this condition typically exhibit a marked fear or anxiety about one or more social situations in which the person is exposed to possible scrutiny by others. Examples include social interactions (e.g., having a conversation), being observed (e.g., eating or drinking), or performance in front of others (e.g., giving a speech). Typically, an individual with this condition (i) fears that he or she will act in a way, or show anxiety symptoms that will be negatively evaluated (i.e., be humiliating, embarrassing, lead to rejection, or offend others); (ii) the social situations almost invariably provoke immediate fear or anxiety; (iii) the social situations are avoided or endured with intense fear or anxiety; and (iv) the fear or anxiety is out of proportion to the danger posed by the social situation. In children, the fear or anxiety may be expressed by crying, tantrums, freezing, clinging, shrinking or refusal to speak in social situations. The fear, anxiety, and avoidance can cause clinically significant distress or impairment in social, occupational, or other important areas of functioning.

As used herein, the terms “treat,” “treating,” or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of inflammation). The term “therapeutic” includes the effect of mitigating deleterious clinical effects of certain inflammatory processes (i.e., consequences of the inflammation, rather than the symptoms of inflammation). The methods of the invention can be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

By “unipolar depression” or “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, and stroke). Generally, unipolar depression is more severe than dysthymia. The essential feature of a major depressive episode is a period of at least two 15 weeks during which there is either depressed mood or loss of interest or pleasure in nearly all activities. In children and adolescents, the mood may be irritable rather than sad. The episode may be a single episode or may be recurrent. The individual also experiences at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans, or attempts. Each symptom must be newly present or must have clearly worsened compared with the person's pre-episode status. The symptoms must persist for most of the day, nearly every day, for at least two consecutive weeks, and the episode must be accompanied by clinically significant distress or impairment in social, occupational (or academic), or other important areas of functioning (Diagnostic and Statistical Manual of Mental Disorders (OSM IV), American Psychiatric Press, 4th Edition, 1994). Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, Va.; American Psychiatric Association, 2013).

Other features and advantages of the invention will be apparent from the following Detailed Description, Examples, Figure, and Claims.

### DETAILED DESCRIPTION

The disclosure provides new methods of treating psychological conditions, neurological injuries, pain, cephalic pain (e.g., headache), inflammatory conditions, and anxiety in a subject by utilizing intravenous psilocybin or psilocin infusion formulations. The psilocybin or psilocin, or a pharmaceutically acceptable salt thereof, infusion may be administered as a monotherapy. The psilocybin or psilocin infusion, or pharmaceutically acceptable salt thereof, may be administered in combination with another therapeutic agent, such as an antiemetic, a benzodiazepine, and/or an anti-inflammatory agent.

### Psychological Conditions

Disclosed herein are methods of treating psychological conditions. The psychological condition may be any psychological condition described herein. In some embodiments the psychological condition is depression, anxiety, addiction, post-traumatic stress disorder (PTSD), an eating disorder, or compulsive behavior. In some embodiments, the psychological condition may be depression. The psychological condition may also be anxiety. The anxiety may be experienced by a subject who is receiving palliative care or is enrolled in a hospice program. In certain embodiments, the subject who is experiencing anxiety has symptoms such as hypervigilance, fatigue, racing thoughts, irritability, excessive worry, and/or fear.

A subject may be diagnosed with a psychological condition by a clinician, a physician, or a therapist. The subject may be diagnosed with a psychological condition by evaluation of the subject's symptoms by a physician, clinician, or therapist, based on a physical examination. For example, a blood test may be used to evaluate blood concentration levels of certain biomarkers such as hormones, calcium, vitamin D, electrolytes, and iron in diagnosing depression. Additionally, or alternatively, for patients with a possible depression condition a depression screening test may be performed by the physician, clinician, or therapist to aid in the diagnosis of depression. The depression screening test may be the Patient Health Questionnaire-9 (PHQ-9), the Beck Depression Inventory (BDI), the Zung Self-Rating Depression Scale, the Center for Epidemiological Studies Depression Scale (CES-D), the Hamilton Rating Scale for Depression (HRSD), or the Montgomery-Asberg Depression Rating Scale (MADRS-C). In some embodiments, the methods described herein may be used to treat psychosomatic pain conditions. In some embodiments, the psychosomatic pain condition may be fibromyalgia, chronic fatigue, migraines, or back pain.

In some embodiments, the patient is being treated for depression with the intravenous infusion of psilocybin. In certain embodiments, the patient is being treated for depression with the intravenous infusion of psilocin. The patient may have their symptoms of depression evaluated using a depression screening test. The symptoms of depression may be evaluated by a clinician using the Clinical Global Impression (CGI) rating. The depression screening test may be the Patient Health Questionnaire-9 (PHQ-9), the Beck Depression Inventory (BDI), the Zung Self-Rating Depression Scale, the Center for Epidemiological Studies Depression Scale (CES-D), the Hamilton Rating Scale for Depression (HRSD), and/or the Montgomery-Asberg Depression Rating Scale (MADRS). The patient being treated for depression with the intravenous infusion of psilocybin or psilocin may have their symptoms of depression evaluated using the Montgomery-Asberg Depression Rating Scale (MADRS-C). In some embodiments, the patient may be evaluated using the MADRS-C by a clinician, physician, or third party rater. In certain embodiments, the patient may self-evaluate using the MADRS. The patient's score obtained using the MADRS-C may be decreased compared to the score before treatment. The patient's score may decreased by at least 50% compared to the score before treatment. The patient's score obtained using the MADRS-C may be less than 10. In some embodiments, the decrease in the patient's score using the MADRS-C is decreased for 1 week after treatment. In certain embodiments, the decrease in the patient's score using the MADRS-C is decreased for 4 weeks after treatment. In particular embodiments, the patient's score using the MADRS-C is decreased for more than 4 weeks after treatment.

In certain embodiments, the patient is being treated for anxiety with intravenous infusion of psilocybin. In some embodiments, the patient is being treated for anxiety with intravenous infusion of psilocin. The patient may have their symptoms of anxiety evaluated using an anxiety screening test. The anxiety screening test may be the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the—General Anxiety Disorder-7. In some embodiments, the patient's anxiety score using any one of these screening tests decreases in comparison to the patient's score before receiving treatment. In certain embodiments, the patient's anxiety score using any one of the above screening tests decreases by 50% in comparison to the patient's score before receiving treatment. In particular embodiments, the patient meets fewer criteria for anxiety as described by the Diagnostic and Statistical Manual of Mental Disorders in comparison before receiving treatment.

In one embodiment, the methods of the invention are used to treat psychological conditions, e.g., depression, anxiety, PTSD, an eating disorder, and compulsive behavior, by administering an infusion of psilocybin or psilocin as needed to treat the symptoms associated with the psychological condition. An example of this method of treatment is when a subject is being treated with three infusions of psilocybin or psilocin, e.g., each administration at a free base equivalent rate of between 1 mg/hr and 15 mg/hr of psilocin or an equimolar equivalent of psilocybin over a period of time of between 10 minutes and 1 hour, spaced at least three days, five days, 10 days, or two weeks apart. The intensity and/or frequency of the symptoms associated with the psychological disorder can be reduced following treatment with infused psilocybin or psilocin. For example, the subject can be treated with a free base equivalent of 5.0±1.0 mg of psilocin, or a pharmaceutically acceptable salt thereof, administered over a period of 20 to 30 minutes.

### Neurological Injuries

Also disclosed herein are methods of treating a neurological injury. The neurological injury may be any neurological injury. In some embodiments, the neurological injury is a stroke, a traumatic brain injury, or a spinal cord injury. The methods of treating a neurological injury described herein may reduce acute inflammation. In certain embodiments, hippocampal hyperactivity is reduced. Also, the methods described herein for treating a neurological injury may be administered in combination with a behavioral, physical, or speech therapy.

In particular embodiments, the methods of the invention are used to treat a neurological injury, e.g., stroke, traumatic brain injury, and spinal cord injury, by administering an infusion of psilocybin or psilocin as needed to treat pain, inflammation, and/or other symptoms associated with the neurological injury. An example of this method of treatment is when a subject is being treated with five infusions of psilocybin or psilocin, e.g., each administration at a free base equivalent rate of between 1 mg/hr and 15 mg/hr of psilocin or an equimolar equivalent of psilocybin over a period of time of between 10 minutes and 1 hour, spaced at least two days, five days, seven days, 10 days, or two weeks apart. For example, the subject can be treated with a free base equivalent of 5.0±1.0 mg of psilocin, or a pharmaceutically acceptable salt thereof, administered over a period of 20 to 30 minutes. The intensity and/or frequency of the symptoms associated with the neurological injury can be reduced following treatment with infused psilocybin or psilocin. Furthermore, the treatment can be used to promote neurogenesis and improve cognitive function following a neurological injury.

### Inflammatory Conditions

An inflammatory condition in a subject can be treated with an infusion of psilocybin or psilocin using the methods of the invention. The inflammatory condition to be treated can be a lung inflammation (e.g., chronic obstructive pulmonary disease (COPD)), neuroinflammation (e.g., Alzheimer's disease), chronic inflammation, rheumatoid arthritis, atherosclerosis, psoriasis, type II diabetes, inflammatory bowel disease, Crohn's disease, multiple sclerosis, and/or septicemia.

In one embodiment, inflammation is treated by administering an infusion of psilocybin or psilocin as needed to treat (i) acute attacks of inflammation (e.g., inflammatory bowel disease), or (ii) chronic inflammatory conditions (e.g., arthritis). An example of this method of treatment is when a subject is being treated with three infusions of psilocybin or psilocin, e.g., each administration at a free base equivalent rate of between 1 mg/hr and 15 mg/hr of psilocin or an equimolar equivalent of psilocybin over a period of time of between 10 minutes and 1 hour, spaced at least three days, five days, 10 days, or two weeks apart. For example, the subject can be treated with a free base equivalent of 5.0±1.0 mg of psilocin, or a pharmaceutically acceptable salt thereof, administered over a period of 20 to 30 minutes. The intensity and/or frequency of the inflammation or pain associated with inflammation can be reduced following treatment with infused psilocybin or psilocin.

### Pain

A disorder or condition associated with pain (e.g., acute or chronic pain of known or unknown origin) can be treated with an infusion of psilocybin or psilocin using the methods of the invention. The pain can be chronic pain, which may result, e.g., from post-operative pain, tension headaches, chronic lower back pain, fibromyalgia, nephropathy, multiple sclerosis, shingles, complex regional pain syndrome, cephalic pain, or sciatica. The chronic pain may arise from an operation. The chronic pain may also be pain associated with a particular disease or condition such as nephropathy, multiple sclerosis, shingles, or complex regional pain syndrome. One particular disorder or condition associated with cephalic pain can be treated with an infusion of psilocybin or psilocin using the methods of the invention. As used herein, a disorder or condition associated with cephalic pain is a disorder or condition which has as one of its symptoms cephalic/head pain (e.g., headache). Examples of such disorders or conditions include trigeminal autonomic cephalalgias such as episodic and chronic cluster headache (CH), episodic and chronic paroxysmal hemicrania (PH), and short-lasting unilateral neuralgiform headache attacks with conjunctival injection and tearing (SUNCT). Other examples of disorders or conditions that can be treated according to the present invention include vascular headaches (e.g., migraine headaches), tension headaches, headaches associated with the use of a substance (e.g., triptans such as sumatriptan, benzodiazepines such as alprazolam, analgesics such as ibuprofen, ergots such as ergotamine, opioids such as morphine, recreational drugs such as caffeine, nicotine, alcohol, and hormone replacement therapy containing, for example, estrogen) or its withdrawal. Yet additional examples of disorders or conditions associated with cephalic pain include miscellaneous headache unassociated with a structural lesion, headache associated with a nonvascular intracranial disorder, headache associated with a non-cephalic infection, headache associated with a metabolic disorder, headache associated with a disorder of the cranium, neck, eyes, nose, sinuses, teeth, mouth, or other facial or cranial structure, nerve trunk pain and deafferentiation pain.

In one embodiment, the methods of the invention are used to treat chronic pain, e.g., post-operative pain, tension headaches, chronic lower back pain, fibromyalgia, nephropathy, multiple sclerosis, shingles, complex regional pain syndrome, cephalic pain, or sciatica, by administering an infusion of psilocybin or psilocin as needed to treat acute attacks of cephalic pain during the period in which the psilocybin or psilocin is being administered. An example of this method of treatment is when a subject is being treated with three infusions of psilocybin or psilocin, e.g., each administration at a free base equivalent rate of between 1 mg/hr and 15 mg/hr of psilocin or an equimolar equivalent of psilocybin over a period of time of between 10 minutes and 1 hour, spaced at least three days, five days, 10 days, or two weeks apart. For example, the subject can be treated with a free base equivalent of 5.0±1.0 mg of psilocin, or a pharmaceutically acceptable salt thereof, administered over a period of 20 to 30 minutes. The intensity and/or frequency of the chronic pain can be reduced following treatment with infused psilocybin or psilocin. In certain embodiments, the subject being treated for chronic pain is also administered a medication for pain relief including morphine, hydromorphone, hydrocodone, meperidine, and fentanyl.

### Psilocybin

Psilocybin, also known as 4-phosphoryloxy-N,N-dimethyltryptamine is a compound capable of producing psychedelic effects, which was originally isolated from psilocybin mushrooms. Psilocybin has the following molecular structure:

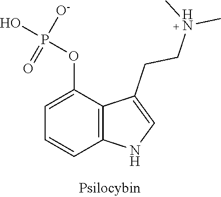

Psilocybin is a prodrug for psilocin, which has a high affinity for the 5-HT2A receptor, also known as the serotonin 2A receptor, which plays a key role in regulating mood, sexual behavior, aggression, impulsivity, cognitive function, appetite, pain, sleep, and memory along with other behaviors. As result, psilocybin binding to the 5-HT2A receptor mimics the binding of serotonin. This disclosure provides methods for treating a patient having a psychological condition or a neurological injury using an intravenous infusion of psilocybin.

In one aspect, the disclosure provides a method for treating a patient with a psychological condition, a neurological injury, an inflammatory condition, or pain using an intravenous infusion of psilocybin or a pharmaceutically acceptable salt thereof. The psilocybin may be administered by intravenous infusion as described here. The rate of infusion may be adjusted so as to minimize adverse side effects and maximize the effectiveness of treatment on the patient's psychological condition. As a result, the subject's intensity of experience is monitored over the course of the infusion such that the rate of infusion can be changed if the subject experiences a negative side effect. Likewise, the subject's plasma concentration of psilocin may be monitored over the course of the infusion. In some embodiments, the plasma concentration of psilocin does not exceed about 40 ng/mL.

The intravenous infusion of psilocybin for the treatment of a disease or condition may be administered at once or twice. In some embodiments, the intravenous infusion of psilocybin may be administered between 2 and 10 times (e.g., 3 times, 4 times, 5 times, 6 times, 7 times, 8 times, 9 times, and 10 times). In certain embodiments, the intravenous infusion of psilocybin may be administered weekly. In some embodiments, the intravenous infusion of psilocybin may be administered every two weeks. In other embodiments, the intravenous infusion of psilocybin may be administered monthly. The intravenous infusion of psilocybin may also be administered every 3 months and the intravenous infusion of psilocybin may be administered every 4 months or 6 months. In particular embodiments, the intravenous infusion of psilocybin may be administered once a year. The intravenous infusion of psilocybin may be administered until the subject's symptoms of their disease or condition are improved compared the subject's symptoms prior to treatment.

### Psilocin

Psilocin, also known as 4-hydroxy-N,N-dimethyltrypamine, is a compound capable of producing psychedelic effects in a subject. Psilocin has the following molecular structure:

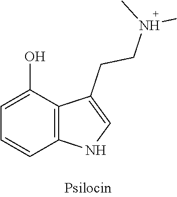

Psilocybin is a prodrug for psilocin, and when administered to a subject, psilocybin is metabolized to form psilocin. Psilocybin undergoes a dephosphorylation reaction resulting in a loss of the phosphate group the hydroxy group. Replacing the negatively charged phosphate group with a hydroxy group, allows psilocin to be more lipid soluble in comparison to psilocybin, and therefore is capable of crossing the blood brain barrier more effectively to elicit a response. Psilocin has a high affinity for the 5-HT2A receptor, which plays a key role in regulating mood, sexual behavior, aggression, impulsivity, cognitive function, appetite, pain, sleep, and memory along with other behaviors. As result, psilocin binding to the 5-HT2A receptor mimics the binding of serotonin. This disclosure provides methods for treating a patient having a psychological condition or a neurological injury using an intravenous infusion of psilocin.

In one aspect, the disclosure provides a method for treating a patient with a psychological condition, a neurological injury, an inflammatory condition, or pain using an intravenous infusion of psilocin or a pharmaceutically acceptable salt thereof. The psilocin may be administered by intravenous infusion as described herein. The rate of administration of the intravenous psilocin infusion may be configured to vary during the period of administration. The rate of infusion may be adjusted such as to minimize adverse side effects and maximize the effectiveness of treatment on the patient's psychological condition. As a result, the subject's intensity of experience is monitored over the course of the infusion such that the rate of infusion can be changed if the subject experiences a negative side effect. Likewise, the subject's plasma concentration of psilocin may be monitored over the course of the infusion. In some embodiments, the amount of plasma concentration of psilocin does not exceed 40 ng/mL.

The intravenous infusion of psilocin for the treatment of a disease or condition may be administered once or twice. In some embodiments, the intravenous infusion of psilocin may be administered between 2 and 10 times (e.g., 3 times, 4 times, 5 times, 6 times, 7 times, 8 times, 9 times, and 10 times). In certain embodiments, the intravenous infusion of psilocin may be administered weekly. In some embodiments, the intravenous infusion of psilocin may be administered every two weeks. In other embodiments, the intravenous infusion of psilocin may be administered monthly. The intravenous infusion of psilocin may also be administered every 3 months and the intravenous infusion of psilocin may be administered every 4 months or 6 months. In particular embodiments, the intravenous infusion of psilocin may be administered once a year. The intravenous infusion of psilocin may be administered until the subject's symptoms of their disease or condition are improved compared the subject's symptoms prior to treatment.

### Combination Therapies

Optionally the methods for treating a disease or condition described herein can include administering to a patient an intravenous infusion of psilocybin or psilocin, or a pharmaceutically acceptable salt thereof, in combination with one or more additional therapeutic agents, including an antiemetic agent, a benzodiazepine, and anti-inflammatory agents. Additionally, described herein specifically are methods of treating a disease or condition where the disease or condition is chronic pain. When the patient is being treated for chronic pain with the psilocybin or psilocin infusion the patient may also be administered one or more medications for pain relief, including morphine, hydromorphone, hydrocodone, meperidine, and fentanyl

Benzodiazepine

In some embodiments described herein, the intravenous psilocybin or psilocin infusion may be administered to a patient having psychological condition or a neurological injury in need of treatment in combination with a pharmacologically effective amount of benzodiazepine. The benzodiazepine may be formulated in the same composition as the psilocybin or psilocin infusion, or the benzopiazepine may be formulated in a separate composition from the psilocybin or psilocin infusion. In some embodiments, the benzodiazepine and the psilocybin or psilocin infusion are administered concurrently. In certain embodiments, the psilocybin or psilocin infusion and the benzodiazepine are administered separately.

The benzodiazepine may be a 1,4-benzodiazepine, 1,5-benzodiazepine, 2,3-benzodiazepine, triazolobenzodiazepine, imidazobenzodiazepine, oxazolobenzodiazepine, thienodiazepine, thienotriazolodiazepine, thienobenzodiazepine, pyridodiazepine, pyridotriazolodiazepine, pyrralodiazepine, tetrahydroisoquinobenzodiazepine, a benzodiazepine prodrug, diazepam, midazolam, alprazolam, temazepam, clonazepam, or a combination thereof. In some embodiments, the benzodiazepine is lorazepam.

Lorazepam is a benzodiazepine medication sold under various trade names including ATIVAN®, ALMAZINE®, and TAVOR®. Lorazepam has various properties, including acting as a sedative, a hypnotic, an amnesiac, and an anxiolytic. Like other benzodiazepines, lorazepam is generally understood to act primarily by enhancing the effect of gamma-aminobutyric acid (GABA) at the GABAA receptor. As the primary inhibitory neurotransmitter, GABA acts to reduce neuronal excitability throughout the nervous system. Compared to other benzodiazepines such as diazepam (valium), lorazepam is substantially more potent and longer acting. Lorazepam has the following molecular structure:

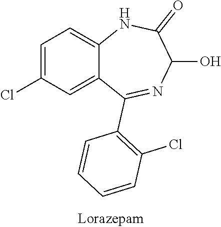 [SMILES: O=C1Nc2ccc(Cl)cc2C(c2ccccc2Cl)=NC1O]

It may be seen, however, that other benzodiazepines may be utilized. In some embodiments described herein, the benzodiazepine is administered in combination with the psilocybin or psilocin infusion such that the ratio of psilocybin or psilocin to benzodiazepine is between 100:1 and 10:1 by weight (e.g., 90:1, 80:1, 70:1, 60:1, 50:1, 40:1, 30:1, 20:1 and 10:1 by weight). The benzodiazepine may be administered in a dosage of between 2 mg and 10 mg (e.g., 2±1 mg, 3±1 mg, 4±1 mg, 5±1 mg, 6±1 mg, 7±1 mg, 8±1, 9±1 mg, and 10±1 mg) in combination with the psilocybin or psilocin infusion. The benzodiazepine administered may be lorazepam. The lorazepam may be administered in a dosage between 2 mg and 4 mg (e.g., 2±1 mg, 3±1 mg, and 4±1 mg). The benzodiazepine administered in combination with the psilocybin or psilocin may be diazepam. The diazepam may be administered in a dosage between 5 mg and 10 mg (e.g., 5±1 mg, 6±1 mg, 7±1 mg, 8±1, 9±1 mg, and 10±1 mg). The benzodiazepine may be administered orally, transmucosally (e.g. nasally, buccally, sublingually, vaginally, ocularly, rectally, etc.) intravenously, by inhalation, intramuscular injection, and any other form of delivery

The benzodiazepine may be administered to the patient to dampen anxiety-producing effects of the psilocybin or psilocin. As a result, the benzodiazepine may decrease the intensity of the experience of the subject being administered the psilocybin or psilocin. Therefore, benzodiazepine may be administered in order to limit, stop, or prevent any negative side effects (such as psilocybin-induced anxiety or psilocin-induced anxiety) from the psilocybin or psilocin that the patient may experience.

Antiemetic Agent

In some embodiments described herein, the intravenous psilocybin or psilocin infusion may be administered to a patient having a disease or condition in need of treatment in combination with a pharmacologically effective amount of an antiemetic agent. The antiemetic agent may be administered to the subject prior to the psilocybin or psilocin infusion. The antiemetic agent may be a non-selective 5-HT antagonist, 5-HT3 receptor antagonist, 5-HT4 receptor agonist, CB1 agonist, D2 receptor antagonist, D3 receptor antagonist, GABA receptor agonist, H1 receptor antagonist, muscarinic acetylcholine receptor antagonist, NK1 receptor antagonist, or a combination thereof. In a preferred embodiment, the antiemetic agent is ondansetron.

Ondansetron is an antiemetic medication sold under the trade names ZOFRAN® and ONDISSOLVE® in various markets. Ondansetron is a 5-HT3 receptor antagonist (a “setron”) generally used in controlling nausea and vomiting in post-operative conditions and in chemotherapy patients, and as well as for various off-label uses. 5-HT3 receptor antagonists bind to and block the 5-HT3 receptor, which is a ligand-gated ion channel found in the vagus nerve and in the area postrema, as well as the in the vomiting center in the medulla oblongata of the brainstem. Synaptic transmission initiated via the 5-HT3 receptor directly mediates the nausea and vomiting reflex. Ondansetron has the following molecular structure:

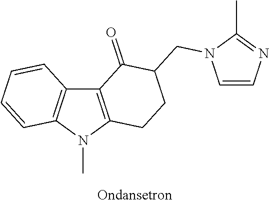 [SMILES: Cc1nccn1CC1CCc2c(c3ccccc3n2C)C1=O]

According to a presently contemplated method of treating a patient, a pharmacologically effective amount of an antiemetic agent may be administered to the patient. The antiemetic agent may be ondansetron. The ondansetron may be administered in a dosage between 4 mg and 8 mg. In some embodiments, the antiemetic agent is administered as a 4 mg intravenous infusion of ondansetron prior to the psilocybin or psilocin infusion. However, it may be seen that in other embodiments, other preparations may be administered to the patient which may vary in dosage form. For example, methods of delivery of the antiemetic agent other than intravenous infusion may be utilized, including but not limited to oral delivery, transmucosal (e.g. nasal, buccal, sublingual, vaginal, ocular, rectal, etc.) delivery, inhalatory delivery, intramuscular injection, and any other form of delivery that may achieve administration to the patient of a pharmacologically effective amount of the antiemetic agent. Likewise, it may also be seen that antiemetic agents other than ondansetron may be utilized, including but not limited to other setrons, or other antiemetic compounds or preparations.

Anti-Inflammatory Agents

In some embodiments described herein, the intravenous psilocybin or psilocin infusion may be administered to a patient having a disease or condition in need of treatment in combination with a pharmacologically effective amount of anti-inflammatory agent. The anti-inflammatory agent may be formulated in the same composition as the psilocybin or psilocin infusion, or the anti-inflammatory agent may be formulated in a separate composition from the psilocybin or psilocin infusion. In some embodiments, the anti-inflammatory agent and the psilocybin or psilocin infusion are administered concurrently. In some embodiments, the psilocybin or psilocin infusion and the anti-inflammatory agent are administered separately. The anti-inflammatory agent may be naproxen, ketoprofen, ibuprofen, tolmetin, etodolac, fenoprofen, diclofenac, flurbiprofen misoprostrol, indomethacin, sulindac, ketorolac, esomeprazole, famotidine, diflunisal, triptan, or sumatriptan. In some embodiments, the anti-inflammatory agent is triptan. In some embodiments, the anti-inflammatory agent is sumatriptan. In some embodiments, the sumatriptan is administered with a dosage of between about 4 mg and about 6 mg (e.g., 4.5 mg, 5.0 mg. 5.5 mg, and 6.0 mg). In particular embodiments, the anti-inflammatory agent is ketorolac. The ketorolc may be administered with a dosage of between about 15 mg and about 30 mg (e.g., 16 mg, 18 mg, 20 mg, 22 mg, 24, mg, 26 mg, 28 mg, and 30 mg). In other particular embodiment, the anti-inflammatory agent is triptan or sumatriptan. The anti-inflammatory agent may be administered orally, transmucosally (e.g. nasally, buccally, sublingually, vaginally, ocularly, rectally, etc.) intravenously, by inhalation, intramuscular injection, or by another method of delivery.

### Pharmaceutical Compositions

For use in the methods and compositions of the invention, the psilocybin or psilocin, or a pharmaceutically acceptable salt thereof, may be contained in any appropriate amount in any suitable carrier substance formulated for intravenous infusion and is generally present in an amount of 1-95% by weight of the total weight of the composition. In particular embodiments, the psilocybin or psilocin is present in an amount of 65-95% by weight of the of the total weight of the composition. In some embodiments, the psilocybin or psilocin for intravenous infusion may be formulated in a saline solution.

The formulation of infusions is well known to those skilled in the art of pharmaceutical formulation. Formulations can be found in Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Compositions for infusion use may be provided in unit dosage forms (e.g., in single-dose ampoules), or in vials containing several doses and in which a suitable preservative may be added. The composition may be in the form of a solution, a suspension, an emulsion, an infusion device, or a delivery device for implantation, or it may be presented as a dry powder to be reconstituted with water or another suitable vehicle before use. Apart from the psilocybin or psilocin, or a pharmaceutically acceptable salt thereof, the composition may include suitable carriers and/or excipients. The psilocybin or psilocin, or a pharmaceutically acceptable salt thereof, may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions of psilocybin or psilocin according to the invention may be in a form suitable for sterile infusion. To prepare such a composition, the psilocybin or psilocin, or a pharmaceutically acceptable salt thereof, is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

The antiemetic, benzodiazepine, anti-inflammatory agents, or pharmaceutically acceptable salts thereof which may be administered in combination with the psilocybin or psilocin infusion and may be formulated in any suitable carrier substance and is generally present in an amount of 1-95% by weight of the total weight of the composition. In some embodiments, the antiemetic, benzodiazepine, anti-inflammatory agents, or pharmaceutically acceptable salts thereof are formulated in a dosage form that is suitable for the oral, parenteral (e.g., intravenously, intramuscularly), rectal, cutaneous, nasal, vaginal, inhalant, skin (patch), or ocular administration route. The antiemetic, benzodiazepine, anti-inflammatory agents, or pharmaceutically acceptable salts thereof may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. Pharmaceutical compositions according to the invention may be formulated to release the antiemetic, benzodiazepine, anti-inflammatory agents, or pharmaceutically acceptable salts thereof, substantially immediately upon administration or at any predetermined time or time period after administration.

Any of the antiemetic, benzodiazepine, anti-inflammatory agents described herein may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

The antiemetic, benzodiazepine, anti-inflammatory agents may also be administered parenterally by injection, infusion, or implantation (intravenous, intramuscular, subcutaneous, or the like) in dosage forms, formulations, or via suitable delivery devices or implants containing conventional, non-toxic pharmaceutically acceptable carriers and adjuvants.

### Dosing Regimens and Intensity Ratings

Administration of an intravenous infusion of psilocybin or psilocin, or a pharmaceutically acceptable salt thereof, may be controlled by a rate of infusion. This is especially preferred in cases in which the subject receives a dosing regimen that at peak plasma levels can result in side effects.

Administration of 0.5 mg of psilocybin or psilocin infused over 30 seconds may result in psilocin plasma concentration levels between 0.5 and 0.6 ng/mL 120 minutes after administration and peak levels at about 9 ng/mL. Administration of 1 mg of psilocybin or psilocin infused over 30 seconds may result in psilocin plasma concentration levels between about 0.8 and 1 ng/mL for 120 minutes after administration and peak levels at about 16 ng/mL. Administration of 2 mg of psilocybin or psilocin infused over 30 seconds may result in psilocin plasma concentration levels greater than about 2 ng/mL 120 minutes after administration and peak levels at about 30 ng/mL.

Also, for example, administration of 0.5 mg of psilocybin or psilocin infused over 30 minutes may result in psilocin plasma concentration levels between 0.5 and 0.6 ng/mL 120 minutes after administration and peak levels at about 1.5 ng/mL. Administration of 1 mg of psilocybin or psilocin infused over 30 minutes may result in psilocin plasma concentration levels between 0.8 and 0.9 ng/mL 120 minutes after administration and peak levels at about 3 ng/mL. Administration of 2 mg of psilocybin or psilocin infused over 30 minutes may result in psilocin plasma concentrations between 1 and 3 ng/mL 120 minutes after administration, and peak levels at about 7 ng/mL. Administration of 6 mg of psilocybin or psilocin infused over 30 minutes may result in psilocin plasma concentration levels between 5 and 7 ng/mL 120 minutes after administration, and peak levels at about 20 ng/mL. Administration of 8 mg of psilocybin or psilocin infused over 30 minutes may result in psilocin plasma concentration between 8 and 9 ng/mL 120 minutes after administration, and peak levels at about 27 ng/mL.

With respect to a longer time of infusion, administration of 0.5 mg of psilocybin or psilocin infused over 60 minutes may result in psilocin plasma concentration levels between 0.5 and 0.7 ng/mL 120 minutes after administration, and peak levels at about 1 ng/mL. Administration of 1 mg of psilocybin or psilocin infused over 60 minutes may result in psilocin plasma concentration levels between 0.8 and 1 ng/mL 120 minutes after administration, and peak levels at about 2 ng/mL. Administration of 2 mg of psilocybin or psilocin infused over 60 minutes may result in psilocin plasma concentration levels between 1 and 3 ng/mL 120 minutes after administration, and peak levels at about 5 ng/mL. Administration of 6 mg of psilocybin or psilocin infused over 30 minutes may result in psilocin plasma concentration levels between 6 and 8 ng/mL 120 minutes after administration, and peak levels at about 14 ng/mL. Administration of 8 mg of psilocybin or psilocin infused over 60 minutes may result in psilocin plasma concentration levels between 8 and 10 ng/mL 120 minutes after administration, and peak levels at about 18 ng/mL.

In terms of the intensity of experience that the patient has as a result of being administered psilocybin or psilocin, administration of 0.5 mg of psilocybin or psilocin infused over 30 seconds may result in an intensity rating of about 2, less than 10 minutes after the beginning of the infusion, and peak intensity at about 7. Administration of 1 mg of psilocybin or psilocin infused over 30 seconds may result in an intensity rating of about 2, less than 30 minutes after the beginning of the infusion, and peak intensity at about 9. Administration of 2 mg of psilocybin or psilocin infused over 30 seconds may result in an intensity rating of about 2, less than 120 minutes after the beginning of the infusion, and peak intensity at about the maximally measurable intensity of 10. Administration of doses at or greater than 3 mg of psilocybin or psilocin infused over 30 seconds may result in an intensity rating of about 2, about 250 minutes after the beginning of the infusion, and in paroxysmal peak drug effects above the intensity scale and potential acute adverse reactions.

When the psilocybin or psilocin is infused for a longer period of time, for example, administration of 0.5 mg of psilocybin or psilocin infused over 30 minutes may result in an intensity rating of about 2, about 30 minutes after the beginning of the infusion, and peak intensity at about 2. Administration of 1 mg of psilocybin or psilocin infused over 30 minutes may result in an intensity rating of about 2, about 40 minutes after the beginning of the infusion, and peak intensity at about 4. Administration of 2 mg of psilocybin or psilocin infused over 30 minutes may result in in an intensity rating of about 2, about 120 minutes after the beginning of the infusion, and peak intensity between 6 and 7. Administration of 6 mg of psilocybin or psilocin infused over 30 minutes may result in an intensity rating of about 2, about 260 minutes after the beginning of the infusion, and peak intensity between 9 and 10. Administration of 8 mg of psilocybin or psilocin infused over 30 minutes may result in an intensity rating of about 2, about 300 minutes after the beginning of the infusion, and peak intensity between 9 and 10.

Also, for example, administration of 0.5 mg of psilocybin or psilocin infused over 60 minutes may result in an intensity rating of always less than 2, and peak intensity less than 2. Administration of 1 mg of psilocybin or psilocin infused over 60 minutes may result in an intensity rating of about 2, about 20 minutes after the beginning of the infusion, and peak intensity between 2 and 3. Administration of 2 mg of psilocybin or psilocin infused over 60 minutes may result in an intensity rating of about 2, about 140 minutes after the beginning of the infusion, and peak intensity at about 5. Administration of 6 mg of psilocybin or psilocin infused over 60 minutes may result in an intensity rating of about 2, about 270 minutes after the beginning of the infusion, and peak intensity between 8 and 9. Administration of 8 mg of psilocybin or psilocin infused over 60 minutes may result in an intensity rating of about 2, about 310 minutes after the beginning of the infusion, and peak intensity between 9 and 10.

In some embodiments, the intravenous psilocybin or psilocin infusion is administered in combination with a benzodiazepine which may result in a dampening and/or shortening of the intensity of the experience of the subject in comparison to when psilocybin or psilocin alone is administered. In certain embodiments, the intravenous psilocybin or psilocin infusion is administered in combination with benzodiazepine which may result in the intensity rating falling below 2 more quickly than the time the intensity rating takes to fall below 2 when only psilocybin or psilocin is administered.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1: Evaluation of the Safety and Efficacy of Intravenous Infusion of Psilocybin for Patients Having Depression

Subjects suffering from depression are treated with an intravenous psilocybin infusion.

To enhance participant safety, all subjects undergo: 1) a preparation session with a staff member prior to dosing; 2) administration of study medications in an aesthetically pleasing room under the supervision of one attendant with video/audio monitoring by a remote staff member; and 3) two post-dose integration sessions during which participants are encouraged to discuss their intervention experience with the attendant.

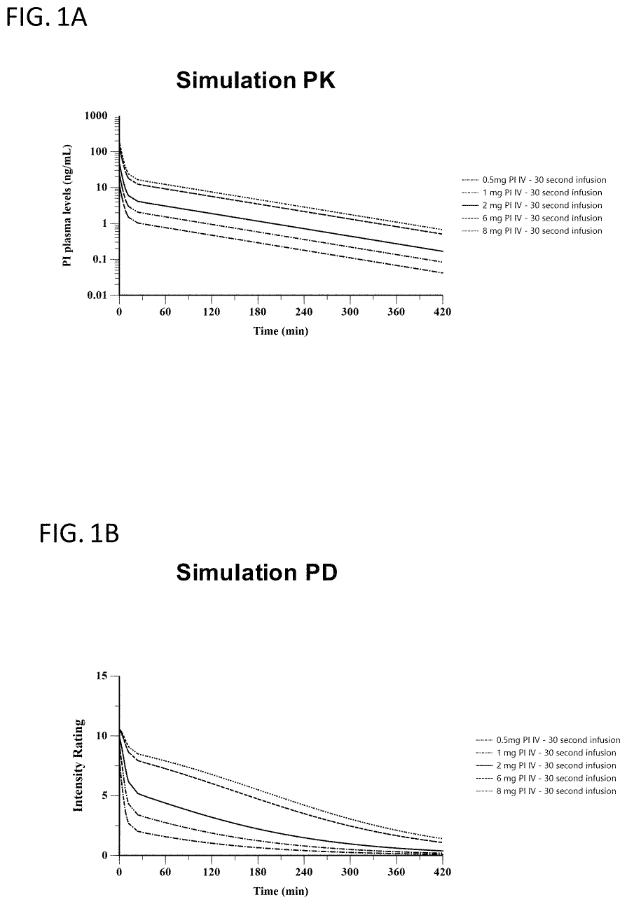

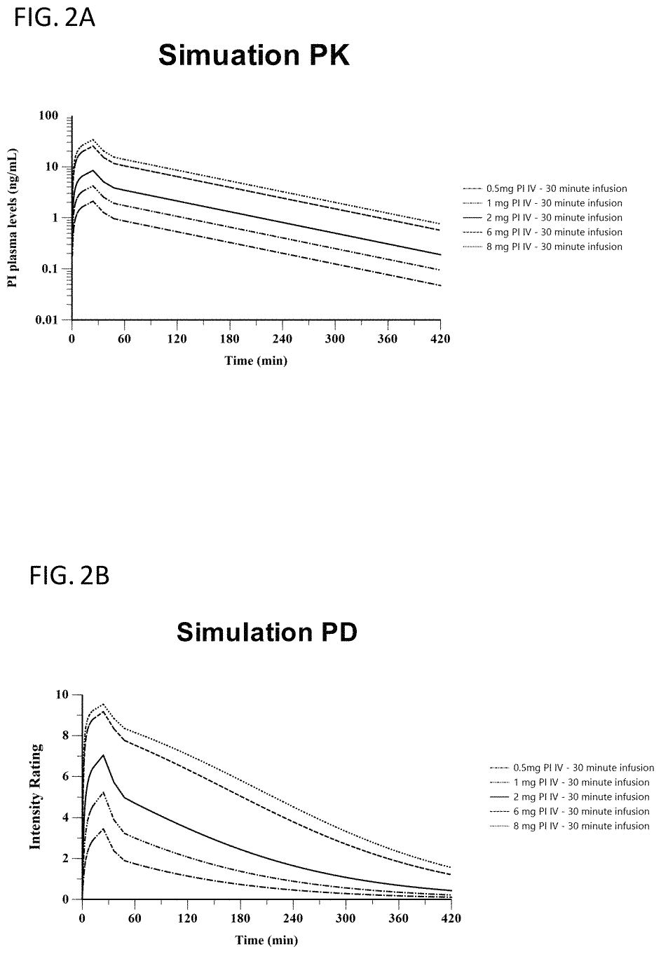

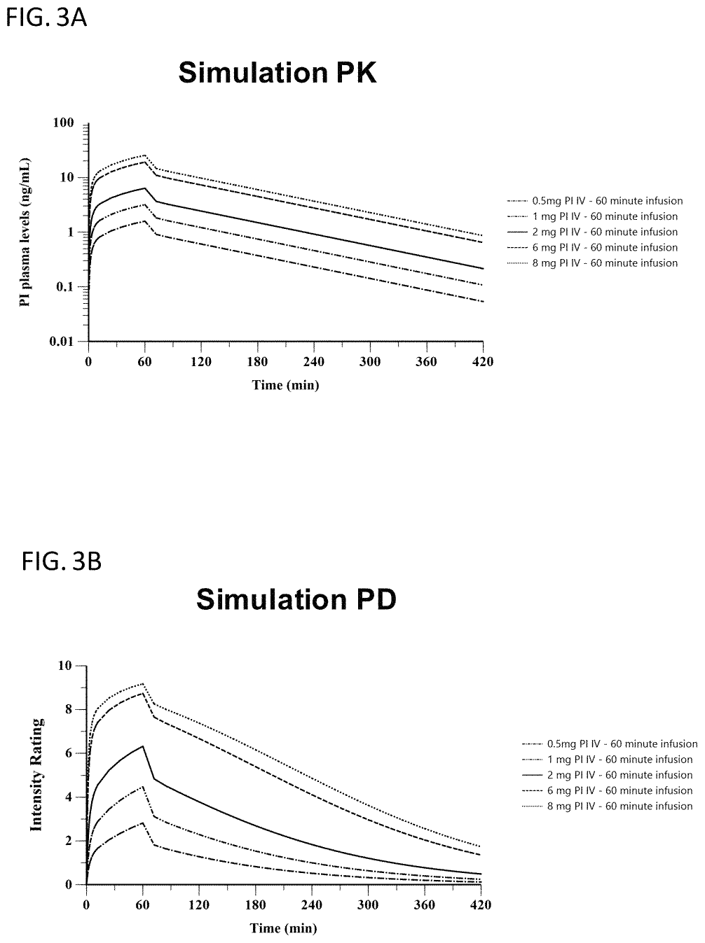

On Day 1, patients are screened to ensure they are between the ages of 25 to 75 years of age, have sustained moderate severe depression symptoms, meet the Diagnostic and Statistical Manual of Mental Disorders-5 criteria for a diagnosis of major depressive disorder and are currently experiencing a major depressive episode of at least a 60-day duration at the time of screening, are able to provide informed consent, are able to stop standard of care treatment for a wash-out period, and have an identified support person and agree to be accompanied home by that person following dosing. Upon selection from the screening, the baseline depression for each patient is evaluated using the Montgomery-Asberg Depression Rating Scale (MADRS) along with other pre-drug baseline assessments. Psilocybin is then administered intravenously to the patient in a dose infusion rate of between 4 mg/hr and 8 mg/hr over a period of up to 60 minutes, and a Cmax per dosing of about 30 ng/mL. Simulations of possible pharmacokinetic and pharmacodynamic data for an infusion rate of 1 mg, 2 mg, 5 mg, or 8 mg over a period of 30 seconds are shown in FIG. 1A and FIG. 1B. Simulations of possible pharmacokinetic and pharmacodynamic data for an infusion rate of 1 mg, 2 mg, 5 mg, or 8 mg over a period of 30 minutes are shown in FIG. 2A and FIG. 2B. Simulations of possible pharmacokinetic and pharmacodynamic data for an infusion rate of 1 mg, 2 mg, 5 mg, or 8 mg over a period of 1 hour are shown in FIG. 3A and FIG. 3B.

While the subject is being administered the psilocybin infusion, the subject's intensity of experience of the psilocybin is monitored using the Drug Effects Questionnaire (DEQ). The infusion rate of psilocybin is reduced when the subject experiences any effects that the subject deems uncomfortable, including confusion, paranoia, or hallucinations, resulting in a decrease in the subject's intensity of experience of the psilocybin. The subject's plasma concentration of psilocin is also monitored while the psilocybin infusion in being administered.

Following administration of the psilocybin, a post-drug assessment is performed. On Day 2, the patient attends a telemedicine appointment for integration and support, and assessments are performed via an online platform. On Day 8, the patient attends a telemedicine appointment for integration and support, assessments are performed via an online platform, and a MADRS-C evaluation is conducted by a third party rater. On Day 30, the patient attends a telemedicine appointment for integration and support, assessments are performed via an online platform, and a MADRS-C evaluation is conducted by a third party rater.

Primary outcome measures are the score of depression severity as measured by the MADRS-C 1 week and 4 weeks after receiving treatment. Secondary outcome measures are a sustained depressive symptom response defined as a 50% reduction from Baseline MADRS score at all post-dose assessments, sustained depressive symptom remission defined as a central rater MADRS total score of at all post-dose assessments, and an evaluation of the effects of psilocybin therapy on the patient quality of life (EQ-5D-3L), anxiety (GAD-7), PHQ-9, positive effect, and stress scale. Patients are also evaluated for changes in serum biomarkers of inflammation, specifically: Tumor Necrosis Factor-Alpha (TNF-α), interleukin (IL)-6, IL-10, IL-1β, C-reactive Protein (CRP), and Macrophage Migration Inhibitory Factor (MIF) at baseline and various timepoints. Patients are also evaluated for changes in biomarkers of metabolic and cardiovascular function, specifically HDL cholesterol, LDL cholesterol, triglycerides, fasting blood glucose, insulin, blood pressure (systolic/diastolic), at baseline, and various timepoints

Patients are evaluated for a variety of psychometrics related to mood, affect, and outlook, including trait and state predictors of dose response. Patients are evaluated for dose response in relation to atypical vs. melancholic features presented by the patient (i.e. BMI, interpersonal (rejection) sensitivity, rumination, appetite, hyper/hyposomnia, leaden paralysis, etc.) as evaluated by [TBD] depressive subtype analysis. Lastly, patients are evaluated by ambulatory assessments and digital biomarkers via mobile health adjuncts.

The psilocybin infusions can reduce symptoms of depression in subjects suffering from depression or a condition associated with depression.

### Example 2: Treatment of Patients Having Anxiety with Intravenous Infusion of Psilocybin

Subjects suffering from anxiety are treated with an intravenous infusion of psilocybin. The subject is first diagnosed with anxiety by a clinician by using a physical exam, using the criteria listed in the Diagnostic and Statistical Manual of Mental Disorders (DSM-5), or conducting an anxiety screening test such as the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the General Anxiety Disorder-7. The subjects are administered a continuous intravenous dosage of psilocybin with an infusion rate of between 1 mg/hr and 15 mg/hr over a time period of no more than one hour. The subject is administered a dosage of psilocybin of up to 2 and 3 times a week for a period of 4 weeks. After 4 weeks, the subject's symptoms associated with anxiety are evaluated using the criteria listed in the Diagnostic and Statistical Manual of Mental Disorders (DSM-5), or the score received on the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the Patient Health Questionnaire-9. The psilocybin infusions can reduce anxiety in subjects suffering from anxiety or a condition associated with anxiety.

### Example 3: Treatment of Patients Having a Psychosomatic Condition with Intravenous Infusion of Psilocybin

Subjects suffering from a somatic disorder experiencing a psychosomatic condition are treated with an intravenous infusion of psilocybin. The subject is first diagnosed by a clinician with somatic disorder as experiencing a psychosomatic condition including fibromyalgia, back pain, migraine, and chronic fatigue syndrome after having first received a physical examination to rule out physical causes for the symptoms experienced by the subject. The subject is also evaluated using the criteria listed in the Diagnostic and Statistical Manual for Mental Disorders (DSM-5) for a somatic disorder. The subject is administered a continuous intravenous dosage of psilocybin at a rate of between 1 mg/hr and 15 mg/hr over a time period of no more than an hour. The subject is administered the intravenous infusion of psilocybin 2 times a week for a period of 3 weeks. The subject's psychosomatic symptoms are then evaluated by a clinician in terms of intensity and frequency of the symptoms that the subject is experiencing currently compared to before receiving treatment. The psilocybin infusions can reduce the intensity and frequency of psychosomatic symptoms in subjects suffering from a psychosomatic condition.

### Example 4: Treatment of Patients Having Post-Traumatic Stress Disorder with Intravenous Infusion of Psilocybin

Subjects suffering from post-traumatic stress disorder (PTSD) are treated with an intravenous infusion of psilocybin. The subject is first diagnosed by a clinician with post-traumatic stress disorder using a physical exam and the criteria listed in the Diagnostic and Statistical Manual for Mental Disorders (DSM-5). The subjects are administered a continuous intravenous dosage of psilocybin at a rate of between 1 mg/hr and 15 mg/hr over a time period of no more than an hour. The intensity of the psilocybin experienced by the subject is rated by the subject and is monitored throughout administration of the psilocybin infusion using the Drug Effects Questionnaire (DEQ). The subjects are simultaneously administered the benzodiazepine lorazepam in the same intravenous formulation as the psilocybin in a dosage of 2 mg to 4 mg. The benzodiazepine is administered in order to lower the intensity of the psilocybin that the subject experiences as reported by the DEQ. The subject is administered the intravenous infusion of psilocybin and lorazepam up to 3 times a week for one week.

One week after the first administration of psilocybin, the subject's symptoms associated with post-traumatic stress disorder are evaluated by a clinician using the criteria listed in the DSM-5. In particular, the subject's anxiety level is assessed using an anxiety screening test such as the Zung Self-Rating Anxiety Scale, the Hamilton Anxiety Scale, the Beck Anxiety Inventory, or the General Anxiety Disorder-7. Additionally, as a result of receiving treatment, the subject is capable of adaptive reconsolidation of the subject's traumatic memory, resulting in a reduction in anxiety, depression, aggression, and/or hypervigilance. As a result of the administration of the psilocybin infusion in combination with lorazepam, the subject can experience less anxiety in comparison the amount of anxiety experienced before treatment. Based on the subject's symptoms associated with post-traumatic stress disorder after receiving treatment in comparison to before receiving treatment, the clinician may recommend continued treatment. The psilocybin infusions can reduce the intensity and frequency of PTSD symptoms (e.g., anxiety or depression) in subjects suffering from PTSD.

### Example 5: Treatment of Patients Having a Traumatic Brain Injury with Intravenous Infusion of Psilocybin

Subjects suffering from a traumatic brain injury are treated with an intravenous infusion of psilocybin. The subjects are first diagnosed by a clinician with a traumatic brain injury using a physical exam. The subjects are administered an intravenous dosage of psilocybin with an infusion rate of between 1 mg/hr and 4 mg/hr over a time period of 30 minutes to 4 hours. The intensity of the psilocybin experienced by the subject is rated by the subject and is monitored throughout administration of the psilocybin infusion using the Drug Effects Questionnaire (DEQ). The infusion rate of psilocybin is lowered by 25% when the subject experiences any effects that the subject deems uncomfortable, including confusion, paranoia, or hallucinations. The lowering of the infusion rate results in a rapid drop in the intensity experienced by the subject and consequently the uncomfortable side effects subside rapidly. The infusion rate is kept at the lower rate to prevent further onset of any uncomfortable side effects for the duration of treatment. The subject is administered the intravenous infusion of psilocybin up to 3 times a week for 4 weeks. After four weeks, the subject is evaluated by a clinician using a physical exam and brain scans in order to evaluate the injury and acute inflammation.

### Example 6: Treatment of Patients Having a Spinal Cord Injury with Intravenous Infusion of Psilocybin

Subjects suffering from a spinal cord injury are treated with an intravenous infusion of psilocybin. The subject is first diagnosed with a spinal cord injury by a physical exam including an MRI or CT scan. The subject is administered an intravenous dosage of psilocybin with an infusion rate of between 1 mg/hr and 4 mg/hr over a time period of between 30 minutes and 4 hours, 3 times a week for a period of 4 weeks. After 4 weeks, the subject is reevaluated by clinician including a physical exam to identify any physical improvements resulting from receiving treatment. The subject's symptoms associated with the spinal cord injury are evaluated and compared to the subject's symptoms before receiving treatment. The subject's pain before and after treatment is evaluated using a numerical pain scale, a Wong-Baker faces pain scale, a FLACC pain scale, a CRIES pain scale, a COMFORT pain scale, a McGill pain scale, a color analog pain scale, a Mankoski pain scale, a Brief Pain Inventory, or a Descriptor Differential Scale of Pain Intensity. The subject's impairment is rated before and after treatment using the Asia Impairment Scale (AIS) or the International Standards for Neurological Classification of Spinal Cord Injury ISNCSCI. The subject is also evaluated in terms of motor and sensory capabilities before and after receiving treatment. The psilocybin infusions can reduce pain in subjects suffering from a spinal cord injury.

### Example 7: Treatment of Patients Having an Eating Disorder with Intravenous Infusion of Psilocybin

Subjects suffering from an eating disorder are treated with an intravenous infusion of psilocybin. The subjects are first diagnosed by a clinician with an eating disorder including anorexia nervosa, bulimia nervosa, and binge eating disorder as having met the criteria listed in the Diagnostic and Statistical Manual for Mental Disorders (DSM-5). The subjects are administered an intravenous dosage of psilocybin with an infusion rate of between 1 mg/hr and 4 mg/hr over a time period of between 30 minutes and 4 hours. The intensity of the psilocybin experienced by the subject is rated by the subject and is monitored throughout administration of the psilocybin infusion. The psilocybin infusion is stopped when the patient experiences a negative side effect including vomiting, muscle weakness, dizziness, paranoia, or frightening hallucinations. As a result of the stopping the infusion of psilocybin, the intensity experienced by the subject drops quickly, resulting in ending the negative side effects in less than 10 minutes. After the negative side effects have subsided, intravenous administration of psilocybin is restarted but now with a lower rate of infusion in comparison to before experiencing the negative side effect. The subject is administered the intravenous infusion of psilocybin and lorazepam up to 3 times a week for two weeks. Two weeks after beginning treatment with psilocybin the subject is evaluated by a clinician to identify any changes in the subject's symptoms associated with an eating disorder. The subject may experience fewer of the symptoms described by the criteria for being diagnosed with an eating disorder as described in the DSM-5 in comparison to the subject's symptoms before receiving treatment. The subject may experience weight gain, fewer purging events per week, fewer binging events per week, or an increase in daily caloric intake as a result of receiving the intravenous psilocybin infusion.

### Example 8: Treatment of Patients Having Post-Operative Pain with Intravenous Infusion of Psilocybin

Subjects suffering from post-operative pain are treated with an intravenous infusion of psilocybin after receiving an operation. The subjects are administered an intravenous dosage of psilocybin with an infusion rate of between 1 mg/hr and 4 mg/hr over a time period to time between 30 minutes and 4 hours. The subject is administered the intravenous infusion of psilocybin daily for 1 week. The psilocybin infusion is administered in combination with either morphine, hydromorphone, or fentanyl. After one week of being treating with psilocybin subject's pain before and after treatment is evaluated using a numerical pain scale, a Wong-Baker faces pain scale, a FLACC pain scale, a CRIES pain scale, a COMFORT pain scale, a McGill pain scale, a color analog pain scale, a Mankoski pain scale, a Brief Pain Inventory, or a Descriptor Differential Scale of Pain Intensity. Based on the effectiveness of the psilocybin treatment and the subject's current level of pain, the clinician may recommend continued intravenous psilocybin infusion treatment. The psilocybin infusions can reduce the intensity and/or frequency of pain experienced by subjects suffering from post-operative pain.

### Example 9: Simulations of Pharmacokinetic and Pharmacodynamic Results of Psilocybin Infusion

Simulations of possible pharmacokinetic and pharmacodynamic data for an infusion rate of 1 mg, 2 mg, 5 mg, or 8 mg over a period of 30 seconds are shown in FIG. 1A and FIG. 1B. Also, simulations of possible pharmacokinetic and pharmacodynamic data for an infusion rate of 1 mg, 2 mg, 5 mg, or 8 mg over a period of 30 minutes are shown in FIG. 2A and FIG. 2B. Lastly, simulations of possible pharmacokinetic and pharmacodynamic data for an infusion rate of 1 mg, 2 mg, 5 mg, or 8 mg over a period of 1 hour are shown in FIG. 3A and FIG. 3B. From these simulations, the maximum concentration of plasma psilocin (Cmax) was calculated, along with the mean plasma psilocin concentration (Cmean) over a defined window of time, and the time it takes for Cmax to be achieved (Tmax), as shown in Table 1. Additionally, the time at which the intensity rating fell below 2 (T at SE<2), the maximum experienced intensity (SEmax), the mean experienced intensity (SE mean) over a defined window of time, and the time it takes to reach the maximum peak of experienced intensity were calculated, also shown in Table 1.

TABLE 1
Calculated Pharmacokinetic and Pharmacodynamic Data for Psilocybin Infusion

| | Plasma Psilocin Pharmacokinetics Simulations | | | | | Acute Effects PK/PD Dynamics Simulations | | | | | |
|---|---|---|---|---|---|---|---|---|---|---|---|
| | | | | | *Time | | | | | SE | *Time |
| | | | AUC | C | for | | T at | SEmax | AUC | mean | for SE |
| | Tmax | Cmax | (min*ng/ | mean | Cmean | Tmax | SE < 2 | (scale | min* | (scale | mean |
| | (min) | (ng/ml) | ml) | (ng/ml)* | (min) | (min) | (min) | 0-10) | SE | 0-10)* | (min) |
| 0.5 mg PY 30 | 0.5 | 7.8 | 132 | 1.7 | 0-30 | 0.5 | 9 | 6.9 | 180 | 2.0 | 0-30 |
| second infusion | | | | | | | | | | | |
| 1 mg PY 30 | 0.5 | 15.7 | 265 | 3.3 | 0-30 | 0.5 | 24 | 9.0 | 334 | 3.4 | 0-30 |
| second infusion | | | | | | | | | | | |
| 2 mg PY 30 | 0.5 | 31.4 | 530 | 6.7 | 0-30 | 0.5 | 108 | 9.9 | 599 | 5.2 | 0-30 |
| second infusion | | | | | | | | | | | |
| 6 mg PY 30 | 0.5 | 94.1 | 1589 | 20.0 | 0-30 | 0.5 | 244 | 10.0 | 1350 | 8.4 | 0-30 |
| second infusion | | | | | | | | | | | |
| 8 mg PY 30 | 0.5 | 125.5 | 2118 | 26.6 | 0-30 | 0.5 | 280 | 10.0 | 1616 | 9.0 | 0-30 |
| second infusion | | | | | | | | | | | |
| 0.5 mg PY 30 | 30 | 1.7 | 132 | 1.0 | 0-60 | 30 | 0 | 2.2 | 187 | 1.4 | 0-60 |
| minute infusion | | | | | | | | | | | |
| 1 mg PY 30 | 30 | 3.3 | 264 | 2.0 | 0-60 | 30 | 42 | 3.9 | 356 | 2.5 | 0-60 |
| minute infusion | | | | | | | | | | | |
| 2 mg PY 30 | 30 | 6.7 | 527 | 4.0 | 0-60 | 30 | 123 | 6.3 | 651 | 4.3 | 0-60 |
| minute infusion | | | | | | | | | | | |
| 6 mg PY 30 | 30 | 20.0 | 1582 | 12.0 | 0-60 | 30 | 260 | 9.5 | 1462 | 8.0 | 0-60 |
| minute infusion | | | | | | | | | | | |
| 8 mg PY 30 | 30 | 26.6 | 2109 | 15.9 | 0-60 | 30 | 295 | 9.8 | 1736 | 8.7 | 0-60 |
| minute infusion | | | | | | | | | | | |
| 0.5 mg PY 1 | 60 | 1.1 | 131 | 0.7 | 0-120 | 60 | 0 | 1.6 | 187 | 1.0 | 0-120 |
| hour infusion | | | | | | | | | | | |
| 1 mg PY 1 | 60 | 2.3 | 262 | 1.4 | 0-120 | 60 | 66 | 2.9 | 360 | 1.8 | 0-120 |
| hour infusion | | | | | | | | | | | |
| 2 mg PY 1 | 60 | 4.6 | 525 | 2.8 | 0-120 | 60 | 139 | 4.9 | 666 | 3.3 | 0-120 |
| hour infusion | | | | | | | | | | | |
| 6 mg PY 1 | 60 | 13.7 | 1574 | 8.3 | 0-120 | 60 | 275 | 8.7 | 1529 | 6.9 | 0-120 |
| hour infusion | | | | | | | | | | | |
| 8 mg PY 1 | 60 | 18.3 | 2098 | 11.0 | 0-120 | 60 | 311 | 9.3 | 1821 | 7.8 | 0-120 |
| hour infusion | | | | | | | | | | | |
| 25 mg-eq PY | 121.8 | 16 | 4635 | 10.4 | 60--360 | 114.4 | 617 | 8.784 | 4087 | 7.7 | 60--360 |
| Oral dose | | | | | | | | | | | |
| 37.5 mg-eq PY | 121.8 | 26.0 | >7000 | ne | 60--360 | ne | ne | ne | ne | ne | 60--360 |
| Oral dose | | | | | | | | | | | |
| 50 mg-eq PY | 123 | 37.6 | >9000 | ne | 60--360 | ne | ne | ne | ne | ne | 60--360 |
| Oral dose | | | | | | | | | | | |

SE: Subjective Effects scale 0-10
ne: not estimated

### Example 10: Treatment of Patients Having Depression with Intravenous Infusion of Psilocin

Subjects suffering from depression are treated with an intravenous infusion of psilocin. The subject is first diagnosed with depression by a clinician by using a physical exam, using the criteria listed in the Diagnostic and Statistical Manual of Mental Disorders (DSM-5), or conducting an depression screening test such as the Patient Health Questionnaire-9 (PHQ-9), the Beck Depression Inventory (BDI), the Zung Self-Rating Depression Scale, the Center for Epidemiological Studies Depression Scale (CES-D), the Hamilton Rating Scale for Depression (HRSD), and/or the Montgomery-Asberg Depression Rating Scale (MADRS). The subjects are administered a continuous intravenous dosage of psilocin with an infusion at a free base equivalent rate of between 4 mg/hr and 8 mg/hr over a time period of no more than one hour. The intensity of the psilocin experienced by the subject is rated by the subject and is monitored throughout administration of the psilocin infusion using the Drug Effects Questionnaire (DEQ). The infusion rate of psilocin is lowered by 20% when the subject experiences any effects that the subject deems uncomfortable, including confusion, paranoia, or hallucinations. The lowering of the infusion rate results in a rapid drop in the intensity experienced by the subject and consequently the uncomfortable side effects subside rapidly. The infusion rate is kept at the lower rate to prevent further onset of any uncomfortable side effects for the duration of treatment. The subject is administered a dosage of psilocin of up to 3 to 4 times a week for a period of 2 weeks. After 2 weeks, the subject's symptoms associated with depression are evaluated using the criteria listed in the Diagnostic and Statistical Manual of Mental Disorders (DSM-5), or the score received on the Patient Health Questionnaire-9 (PHQ-9), the Beck Depression Inventory (BDI), the Zung Self-Rating Depression Scale, the Center for Epidemiological Studies Depression Scale (CES-D), the Hamilton Rating Scale for Depression (HRSD), and/or the Montgomery-Asberg Depression Rating Scale (MADRS). The psilocin infusions can reduce depression in subjects suffering from depression or a condition associated with depression.

### Example 11: Treatment of Patients Having Anxiety with Intravenous Infusion of Psilocin

Subjects suffering from anxiety are treated with an intravenous infusion of psilocin. The subject is first diagnosed with anxiety by a clinician by using a physical exam, using the criteria listed in the Diagnostic and Statistical Manual of Mental Disorders (DSM-5), or conducting an anxiety screening test such as the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the General Anxiety Disorder-7. The subjects are administered a continuous intravenous dosage of psilocin with an infusion at a free base equivalent rate of between 1 mg/hr and 15 mg/hr over a time period of no more than one hour. For example, the subject can be treated with a free base equivalent of 5.0±1.0 mg of psilocin, or a pharmaceutically acceptable salt thereof, administered over a period of 20 to 30 minutes. The intensity of the psilocin experienced by the subject is rated by the subject and is monitored throughout administration of the psilocin infusion using the Drug Effects Questionnaire (DEQ). The subjects are simultaneously administered the benzodiazepine diazepam in the same intravenous formulation as the psilocin in a dosage of 5 mg to 10 mg. The benzodiazepine is administered in order to lower the intensity of the psilocin that the subject experiences as reported by the DEQ. The subject is administered a dosage of psilocin of up to 2 and 3 times a week for a period of 4 weeks. After 4 weeks, the subject's symptoms associated with anxiety are evaluated using the criteria listed in the Diagnostic and Statistical Manual of Mental Disorders (DSM-5), or the score received on the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the Patient Health Questionnaire-9. The psilocin infusions can reduce anxiety in subjects suffering from anxiety or a condition associated with anxiety.

### Example 12: Treatment of Patients Having a Traumatic Brain Injury with Intravenous Infusion of Psilocin

Subjects suffering from a traumatic brain injury are treated with an intravenous infusion of psilocin. The subjects are first diagnosed by a clinician with a traumatic brain injury using a physical exam. The subjects are administered an intravenous dosage of psilocin with an infusion at a free base equivalent rate of between 1 mg/hr and 15 mg/hr over a time period of 30 minutes to 4 hours. For example, the subject can be treated with a free base equivalent of 5.0±1.0 mg of psilocin, or a pharmaceutically acceptable salt thereof, administered over a period of 20 to 30 minutes. The intensity of the psilocin experienced by the subject is rated by the subject and is monitored throughout administration of the psilocin infusion using the Drug Effects Questionnaire (DEQ). The subjects are administered the benzodiazepine lorazepam when the subject experiences any effects that the subject deems uncomfortable, including confusion, paranoia, or hallucinations. The benzodiazepine is simultaneously administered in the same intravenous formulation as the psilocin in a dosage of 2 mg to 4 mg. The benzodiazepine is administered in order to lower the intensity of the psilocin that the subject experiences as reported by the DEQ. The subject is administered the intravenous infusion of psilocin up to 3 times a week for 6 weeks. After 6 weeks, the subject is evaluated by a clinician using a physical exam and brain scans in order to evaluate the injury and acute inflammation.

### Example 13: Treatment of Patients Having Post-Operative Pain with Intravenous Infusion of Psilocin

Subjects suffering from post-operative pain are treated with an intravenous infusion of psilocin after receiving an operation. The subjects are administered an intravenous dosage of psilocin with an infusion at a free base equivalent rate of between 1 mg/hr and 15 mg/hr over a time period to time between 30 minutes and 4 hours. For example, the subject can be treated with a free base equivalent of 5.0±1.0 mg of psilocin, or a pharmaceutically acceptable salt thereof, administered over a period of 20 to 30 minutes. The subject is administered the intravenous infusion of psilocin 2 to 3 times a week for 4 weeks. The psilocin infusion is administered in combination with either morphine, hydromorphone, or fentanyl. After 4 weeks of being treating with psilocin, the subject's pain before and after treatment is evaluated using a numerical pain scale, a Wong-Baker faces pain scale, a FLACC pain scale, a CRIES pain scale, a COMFORT pain scale, a McGill pain scale, a color analog pain scale, a Mankoski pain scale, a Brief Pain Inventory, or a Descriptor Differential Scale of Pain Intensity. Based on the effectiveness of the psilocin treatment and the subject's current level of pain, the clinician may recommend continued intravenous psilocin infusion treatment. The psilocin infusions can reduce the intensity and/or frequency of pain experienced by subjects suffering from post-operative pain.

### Example 14: Treatment of Patients Having Post-Traumatic Stress Disorder with Intravenous Infusion of Psilocin

Subjects suffering from post-traumatic stress disorder (PTSD) are treated with an intravenous infusion of psilocin. The subject is first diagnosed by a clinician with post-traumatic stress disorder using a physical exam and the criteria listed in the Diagnostic and Statistical Manual for Mental Disorders (DSM-5). The subjects are administered a continuous intravenous dosage of psilocin with an infusion at a free base equivalent rate of between 1 mg/hr and 15 mg/hr over a time period of no more than an hour. For example, the subject can be treated with a free base equivalent of 5.0±1.0 mg of psilocin, or a pharmaceutically acceptable salt thereof, administered over a period of 20 to 30 minutes. The subject is administered the intravenous infusion of psilocin daily for one week.

One week after the first administration of psilocin, the subject's symptoms associated with post-traumatic stress disorder are evaluated by a clinician using the criteria listed in the DSM-5. In particular, the subject's anxiety level is assessed using an anxiety screening test such as the Zung Self-Rating Anxiety Scale, the Hamilton Anxiety Scale, the Beck Anxiety Inventory, or the General Anxiety Disorder-7. Additionally, as a result of receiving treatment, the subject is capable of adaptive reconsolidation of the subject's traumatic memory, resulting in a reduction in anxiety, depression, aggression, and/or hypervigilance. As a result of the administration of the psilocin infusion in combination with lorazepam, the subject can experience less anxiety in comparison the amount of anxiety experienced before treatment. Based on the subject's symptoms associated with post-traumatic stress disorder after receiving treatment in comparison to before receiving treatment, the clinician may recommend continued treatment. The psilocin infusions can reduce the intensity and frequency of PTSD symptoms (e.g., anxiety or depression) in subjects suffering from PTSD.

### Example 15: Simulations of Pharmacokinetic and Pharmacodynamic Results of Psilocin Infusion

A compartmental PK model approach was utilized to simulate circulating concentrations of psilocin under different dosing levels and infusion periods. A 2-compartmental PK model with first order elimination from the central compartment was fitted to the data using software Phoenix 64 version 8.0.0.3176. This PK model was then used to simulate psilocin concentration-time profiles of several psilocin IV dosing regimens. In Table 2, simulated maximum plasma concentrations (Cmax) (dose and infusion duration) for 1, 2, 4, 5 and 10 mg administered as 60-, 45-, 30-, 20-, 10- and 2 minute IV infusion are presented. In Table, simulated area under the concentration-time curve (AUC) for 1, 2, 4, 5 and 10 mg administered as IV infusion are presented. In

Table 4 the predicted time that plasma concentrations will be maintained between 10 and 20 ng/mL for the same regimens is provided.

TABLE 2
Predicted psilocin maximum plasma concentration (ng/ml)

| | 60-min | 45-min | 30-min | 20-min | 10-min | 2-min |
|---|---|---|---|---|---|---|
| Dose | IV | IV | IV | IV | IV | IV |
| (mg) | Infusion | Infusion | Infusion | Infusion | Infusion | Infusion |
| 1 | 3.18 | 3.70 | 4.62 | 5.92 | 9.24 | 18.3 |
| 2 | 6.36 | 7.40 | 9.26 | 11.8 | 18.5 | 36.6 |
| 4 | 12.7 | 14.8 | 18.5 | 23.7 | 37.0 | 73.1 |
| 5 | 15.9 | 18.5 | 23.2 | 29.6 | 46.2 | 91.4 |
| 10 | 31.8 | 37.0 | 46.3 | 59.2 | 92.4 | 182.8 |

TABLE 3
Predicted AUC_{inf} (min*ng/mL)

| Dose (mg) | IV Infusion |
|---|---|
| 1 | 381 |
| 2 | 761 |
| 4 | 1522 |
| 5 | 1903 |
| 10 | 3806 |

t_{1/2} = 85.8 minutes for all dose groups and infusion durations

TABLE 4
Predicted Time (minutes) maintained between 10 and 20 ng/ml

| | 60-min | 45-min | 30-min | 20-min | 10-min | 2-min |
|---|---|---|---|---|---|---|
| Dose | IV | IV | IV | IV | IV | IV |
| (mg) | Infusion | Infusion | Infusion | Infusion | Infusion | Infusion |
| 1 | 0 | 0 | 0 | 0 | 0 | 3.6 |
| 2 | 0 | 0 | 0 | 8.57 | 10.2 | 4.2 |
| 4 | 26.8 | 31.8 | 30.1 | 16.5 | 9.3 | 7.9 |
| 5 | 46.7 | 46.0 | 30.2 | 20.5 | 19.0 | 18.7 |
| 10 | 93.3 | 87.3 | 86.1 | 86.0 | 85.8 | 85.5 |

The relationship between psilocin plasma concentration and pharmacodynamics (serotonin 2A receptor (5-HT 2AR) occupancy and subjective psychedelic effects) was used to establish a reference plasma concentration. For both receptor saturation and subjective psychedelic effects an Emax model was used to derive effects at a given concentration. For receptor occupancy the following parameters were used: Emax of 76.6%; and EC50 of 1.95 ng/mL. For subjective intensity the following parameters were used: Emax of 10.8% and EC50 of 4.5 ng/mL. Psilocin plasma concentration of 10 ng/mL and 20 ng/mL are associated with 64.1% and 69.8% of 5-HT 2AR receptor saturation, respectively and intensity ratings of 7.5 and 8.8, respectively.

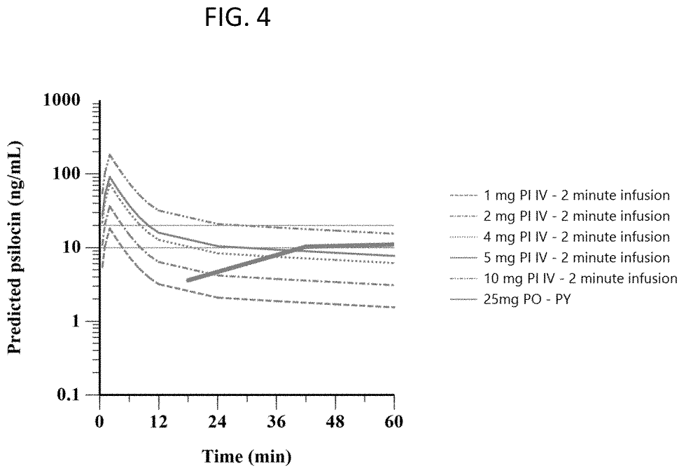

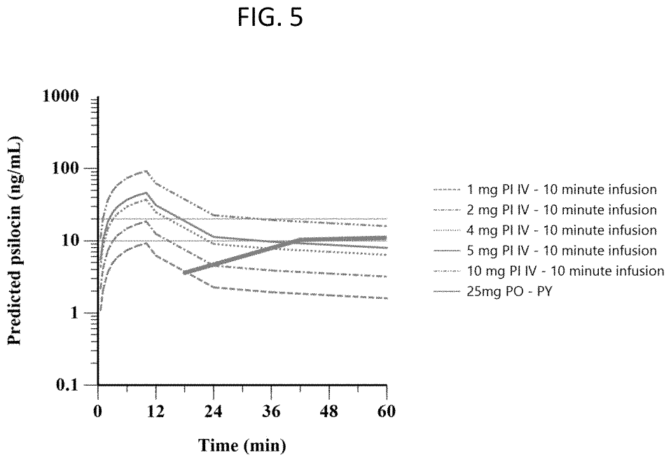

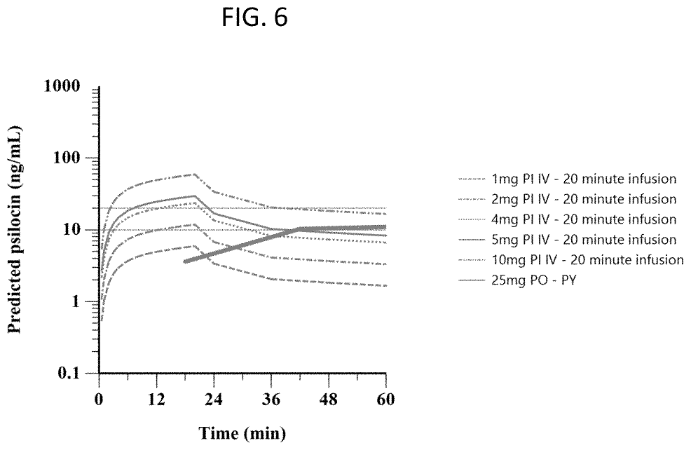

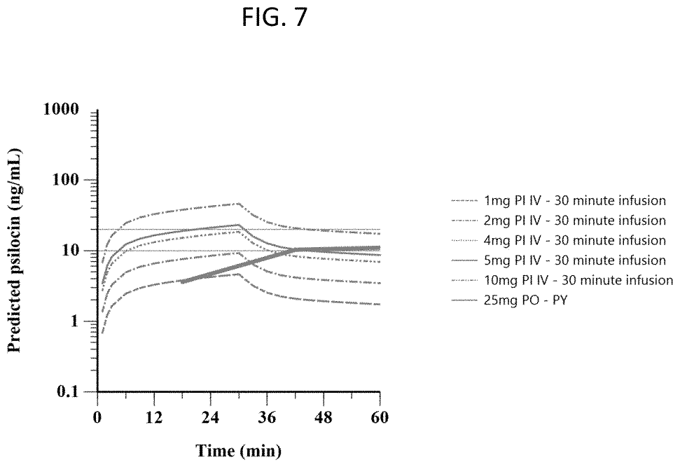

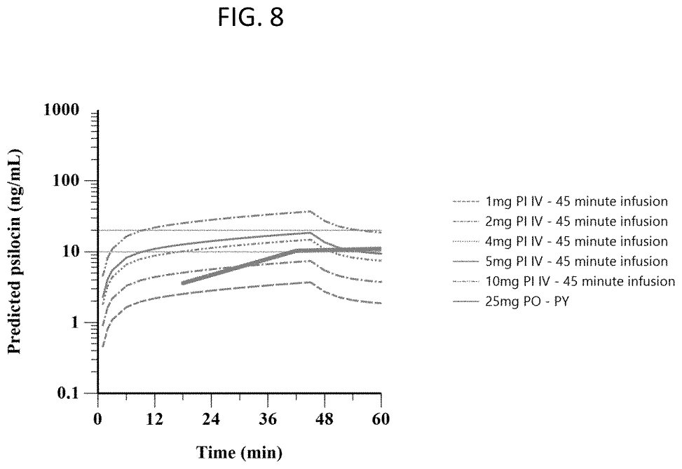

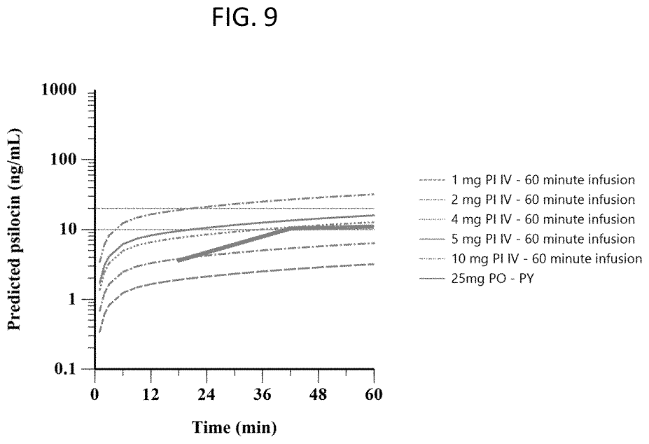

The simulate psilocin concentration-time profiles are depicted in FIGS. 4-9 for 2 min (FIG. 4), 10 min (FIG. 5) and 20 min (FIG. 6). 30-min (FIG. 7); 45 min (FIG. 8) and 60-minutes IV infusion (FIG. 9).

### Example 16: Rat Study of Psilocin in Combination with Lorazepam for the Treatment of Depression

A rat model of depression was used to assess the antidepressant effect of psilocin alone or in combination with lorazepam. WKY rats were divided into 3 groups (n=8 animals per group) and treated with (i) 1 mg/kg of psilocin injected as a bolus dose into the tail vein (PSI); (ii) 1.8 mg/kg of lorazepam injected 30 i.p. minutes prior to a 1 mg/kg tail vein injection of psilocin (P+L); or (iii) saline (SAL; as a control group). The dose of lorazepam was chosen to be a moderate/low non-sedating but anxiolytic dose in rats after consultation with an expert in the field of benzodiazepine's effects in rats.

After injection treatment, rats were maintained in their home cage for three weeks (21 days) prior to testing by forced swim test (FST) as described in Hibicke et al., ACS Chem. Neurosci. 2020, 11, 6, 864-871.

Forced Swim Test methodology: During a pre-exposure, rats were placed into a plastic cylindrical tank (114 cm×30.5 cm) that contained 30 cm of water at 28-30° C. The water depth was such that the rats could not support themselves by touching the bottom of the tank with their hind paws, and their tails could not touch the bottom of the tank while keeping their noses above water. After a 15 min swim, the rats were removed, dried with paper towels, and replaced in their home cages. Fresh water was used for each animal. For the testing, a video camera was mounted to the side of the tank, and the rats were exposed to a 5 min swim under the conditions described above and then removed, dried with paper towels, and returned to their home cages. The 5 min swim was recorded for later scoring for immobility, swimming, and climbing. FST scoring was performed by a trained scorer. Immobility was defined as no active attempts to escape while maintaining a floating posture in which the rats make only the movements necessary to keep their heads above water. Swimming was defined as actively attempting escape with motions directed outward against the wall of the cylinder. Climbing was defined as actively attempting escape with motions directed upward against the wall of the cylinder. Significantly greater immobility than control (SAL) rats indicates depressive-like behavior, and significantly less immobility indicates an antidepressant-like effect.

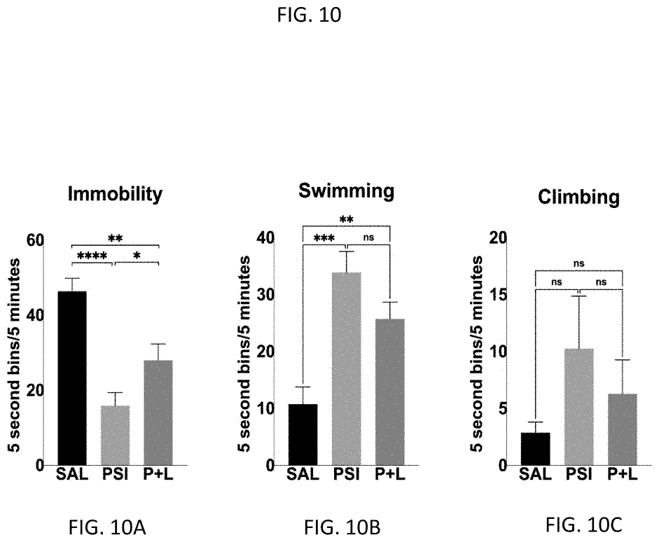

The results are depicted in FIG. 10. Lorazepam pretreatment had a slight but significant effect to blunt the effects of psilocin in the mobility measure, but no significant effect on swimming behavior. There were no statistically significant differences between treatment groups in climbing behaviors.

### CONCLUSIONS

A single bolus of i.v. administered psilocin is as efficacious in producing long-lasting antidepressant-like behaviors in this model as a single i.p. administration of psilocybin of the same dose.

Lorazepam only had a slight, but significant, effect on one outcome measure to reduce efficacy (Immobility), but there was still a robust antidepressant-like effect in the animals treated with the combination. The efficacy of the combination in our rat data indicate that a similar combination in humans will be therapeutically efficacious to treat depression.

The psilocin+lorazepam combination produces long-lasting antidepressant-like effects in this model, nearly as efficacious as psilocybin itself when administered i.p., and psilocin when administered i.v. Our data supports that notion that intravenous psilocin is still therapeutically efficacious following an anxiolytic dose of lorazepam. If translatable to humans, these data suggest that lorazepam can be administered to humans at an anxiolytic level to minimize anxiety associated with psilocin/psilocybin treatment without blocking therapeutic effects of psilocin/psilocybin.

### OTHER EMBODIMENTS

All publications, patents, and patent applications mentioned in this specification are herein incorporated by reference to the same extent as if each independent publication or patent application was specifically and individually indicated to be incorporated by reference. This application claims the benefit of U.S. provisional Ser. Nos. 63/050,428, filed Jul. 10, 2020, and 63/091,068, filed Oct. 13, 2020, each of which is incorporated herein by reference in its entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of treating a disease or condition in a subject in need thereof, the method comprising intravenously administering to the subject a free base equivalent of from 1 mg to 15 mg of psilocin, or a pharmaceutically acceptable salt thereof, over a period of between 1 minute and 60 minutes.

2. The method of claim 1, wherein a free base equivalent of from 4 mg to 15 mg of psilocin, or a pharmaceutically acceptable salt thereof, is administered to the subject over a period of 20 to 60 minutes.

3. The method of claim 2, wherein a free base equivalent of 5.0±1.0 mg of psilocin, or a pharmaceutically acceptable salt thereof, is administered over a period of 20 to 30 minutes.

4. The method of claim 2, wherein a free base equivalent of 7.5±1.0 mg of psilocin, or a pharmaceutically acceptable salt thereof, is administered over a period of 20 to 45 minutes.

5. The method of claim 2, wherein a free base equivalent of 10.0±2.0 mg of psilocin, or a pharmaceutically acceptable salt thereof, is administered over a period of 30 to 60 minutes.

6. The method of claim 1, wherein a free base equivalent of from 1 mg to 5 mg of psilocin, or a pharmaceutically acceptable salt thereof, is administered to the subject over a period of 2 to 20 minutes.

7. The method of claim 6, wherein a free base equivalent of 4.0±1.0 mg of psilocin, or a pharmaceutically acceptable salt thereof, is administered over a period of 5 to 20 minutes.

8. The method of claim 6, wherein a free base equivalent of 3.0±1.0 mg of psilocin, or a pharmaceutically acceptable salt thereof, is administered over a period of 5 to 15 minutes.

9. The method of claim 6, wherein a free base equivalent of 2.0±1.0 mg of psilocin, or a pharmaceutically acceptable salt thereof, is administered over a period of 2 to 10 minutes.

10. A method of treating a disease or condition in a subject in need thereof, the method comprising intravenously administering to the subject a pharmacologically effective amount of (i) psilocybin or psilocin, or a pharmaceutically acceptable salt thereof, and (ii) a benzodiazepine, each in an amount that together is effective for the treatment of the disease or condition.

11. A method of treating a disease or condition in a subject in need thereof, the method comprising administering to the subject a timed intravenous infusion of psilocybin or psilocin, or a pharmaceutically acceptable salt thereof, wherein (i) the timed intravenous infusion is administered at a free base equivalent rate of between 1 mg/hr and 15 mg/hr of psilocin or an equimolar equivalent of psilocybin for a period of time between 10 minutes and 60 minutes; or (ii) the timed intravenous infusion is administered at a free base equivalent rate of between 15 mg/hr and 60 mg/hr of psilocin or an equimolar equivalent of psilocybin for a period of time between 2 minutes and 10 minutes.

12. The method of claim 11, wherein the pharmacologically effective amount of psilocybin or psilocin is administered as a saline solution.

13. The method of any one of claims 1-12, wherein the method comprises further administering to the patient a pharmacologically effective amount of an antiemetic agent.

14. The method of claim 13, wherein the antiemetic agent comprises a non-selective 5-HT antagonist, 5-HT3 receptor antagonist, 5-HT4 receptor agonist, CB1 agonist, D2 receptor antagonist, D3 receptor antagonist, GABA receptor agonist, H1 receptor antagonist, muscarinic acetylcholine receptor antagonist, NK1 receptor antagonist, or a combination thereof.

15. The method of claim 14, wherein the anti-emetic ondansetron is intravenously infused.

16. The method of any one of claims 1-15, wherein the intravenous infusion comprises a pharmacologically effective amount of a benzodiazepine.

17. The method of claim 6, wherein the benzodiazepine is a 1,4-benzodiazepine, 1,5-benzodiazepine, 2,3-benzodiazepine, triazolobenzodiazepine, imidazobenzodiazepine, oxazolobenzodiazepine, thienodiazepine, thienotriazolodiazepine, thienobenzodiazepine, pyridodiazepine, pyridotriazolodiazepine, pyrralodiazepine, tetrahydroisoquinobenzodiazepine, a benzodiazepine prodrug, or a combination thereof.

18. The method of claim 17, wherein the benzodiazepine is lorazepam or diazepam.

19. The method of any one of claims 16-18, wherein the benzodiazepine is administered in a dosage between 2 mg and 10 mg.

20. The method of any one of claims 1-19, wherein the intravenous infusion comprises a pharmacologically effective amount of an anesthetic, a sedative, an antiemetic, an anticonvulsant, an antidepressant, an antimigraine, an antipsychotic, an anxiolytic, and/or an antiparkinson agent.

21. The method of any one of claims 1-20, wherein the intravenous infusion comprises ondansetron or a pharmaceutically acceptable salt thereof.

22. The method of any one of claims 1-21, further comprising administering to the patient a preparation comprising a pharmacologically effective amount of an anti-inflammatory agent.

23. The method of claim 22, wherein the administration of the preparation is an intravenous infusion of ketorolac or pharmaceutically acceptable salt thereof.

24. The method of claim 22, wherein the administration of the preparation is an intramuscular infusion of a pharmacologically effective amount of a triptan or a pharmaceutically acceptable salt thereof.

25. The method of claim 24, wherein the preparation comprises sumatriptan or a pharmaceutically acceptable salt thereof.

26. The method of any one of claims 1-25, wherein the rate of administration of the intravenous infusion comprising a pharmacologically effective amount of psilocybin or psilocin is configured to vary during the period of administration.

27. The method of any one of claims 1-26, wherein the subject's intensity of experience is monitored.

28. The method of claim 27, wherein the rate of administration of the intravenous infusion comprising a pharmacologically effective amount of psilocybin or psilocin is adjusted in response to the subject's intensity of experience.

29. The method of any one of claims 1-28, wherein the patient's plasma concentration of psilocin is monitored.

30. The method of any one of claims 1-29, wherein the intravenous infusion comprising a pharmacologically effective amount of psilocybin or psilocin is administered at least twice over the course of a month.

31. The method of claim 30, wherein the intravenous infusion comprising a pharmacologically effective amount of psilocybin or psilocin is administered between 2 and 10 times over the course of a year.

32. The method of any one of claims 11-31, wherein the timed intravenous infusion of the pharmacologically effective amount of psilocybin or psilocin is administered at a free base equivalent rate of between 4 mg/hr and 15 mg/hr of psilocin or an equimolar equivalent of psilocybin over a period of time of between 10 minutes and 60 minutes.

33. The method of claim 32, wherein a free base equivalent of 4.0±0.5 mg or 5.0±0.5 mg of psilocin or an equimolar equivalent of psilocybin is administered over a period of 20 to 60 minutes.

34. The method of claim 32, wherein a free base equivalent of 10.0±1.0 mg of psilocin or an equimolar equivalent of psilocybin is administered over a period of 45 to 60 minutes.

35. The method of any one of claims 11-31, wherein the timed intravenous infusion of the pharmacologically effective amount of psilocybin or psilocin is administered at a free base equivalent rate of between 15 mg/hr and 30 mg/hr of psilocin or an equimolar equivalent of psilocybin over a period of time of between 2 minutes and 10 minutes.

36. The method of claim 35, wherein a free base equivalent of 1.0±0.5 mg or 2.0±0.5 mg of psilocin or an equimolar equivalent of psilocybin is administered over a period of 2 to 10 minutes.

37. The method of claim 35, wherein a free base equivalent of 4.0±0.5 mg of psilocin or an equimolar equivalent of psilocybin is administered over a period of 5 to 10 minutes.

38. The method of claim 35, wherein a free base equivalent of 5.0±0.5 mg of psilocin or an equimolar equivalent of psilocybin is administered over a period of 10 to 15 minutes.

39. The method of any one of claims 1-38, wherein the condition is a psychological condition.

40. The method of claim 39, wherein the psychological condition is evaluated 1-8 weeks after treatment.

41. The method of claim 40, wherein the psychological condition is evaluated 1 week after treatment.

42. The method of any one of claims 39-41, wherein the psychological condition is depression, anxiety, addiction, post-traumatic stress disorder, an eating disorder, or compulsive behavior.

43. The method of claim 42, wherein the psychological condition is depression.

44. The method of claim 42, wherein the psychological condition is anxiety.

45. The method of claim 44, wherein the anxiety is of a subject receiving palliative care.

46. The method of any one of claims 1-38, wherein the disease or condition is a neurological injury, an inflammatory condition, or pain.

47. The method of claim 46, wherein the disease or condition is an inflammatory condition.

48. The method of claim 47, wherein the inflammatory condition is lung inflammation, neuroinflammation, rheumatoid arthritis, atherosclerosis, psoriasis, type II diabetes, inflammatory bowel disease, Crohn's disease, multiple sclerosis, and/or septicemia.

49. The method of claim 48, wherein the inflammatory condition is chronic obstructive pulmonary disease (COPD), or Alzheimer's disease.

50. The method of claim 46, wherein the disease or condition is a neurological injury.

51. The method of claim 50, wherein the neurological injury is a stroke, a traumatic brain injury, or a spinal cord injury.

52. The method of claim 46, wherein the disease or condition is chronic pain.

53. The method of claim 52, wherein the chronic pain results from post-operative pain, tension headaches, chronic lower back pain, fibromyalgia, nephropathy, multiple sclerosis, shingles, complex regional pain syndrome, cephalic pain, or sciatica.

54. The method of claim 53, wherein the chronic pain condition results from trigeminal autonomic cephalalgia.

55. The method of claim 54, wherein the trigeminal autonomic cephalalgia is selected from the group consisting of episodic and chronic cluster headache (CH), episodic and chronic paroxysmal hemicrania (PH), and short-lasting unilateral neuralgiform headache attacks with conjunctival injection and tearing (SUNCT).

56. The method of claim 55, wherein the trigeminal autonomic cephalalgia is episodic or chronic CH.

57. The method of any one of claims 52-56, further comprising administering to the patient one or more medications for pain relief, comprising morphine, hydromorphone, hydrocodone, meperidine, and fentanyl.

58. The method of any one of claims 46-57, comprising a timed intravenous infusion of the pharmacologically effective amount of psilocybin or psilocin that is administered at a free base equivalent rate of between 1 mg/hr and 15 mg/hr of psilocin or an equimolar equivalent of psilocybin over a period of time of between 10 minutes and 60 minutes.
